(19) United States
(12) Patent Application Publication
(10) Pub. No.: US 2024/0321396 A1
(43) Pub. Date: Sep. 26, 2024

(54) DETECTION OF SOMATIC MUTATIONAL SIGNATURES FROM WHOLE GENOME SEQUENCING OF CELL-FREE DNA

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Jonathan Chee Ming Wan, New York, NY (US); Luis A. Diaz,, JR., New York, NY (US)

(21) Appl. No.: 18/575,530

(22) PCT Filed: Jun. 29, 2022
(86) PCT No.: PCT/US22/35449

§ 371 Date: Dec. 29, 2023

Related U.S. Application Data
(60) Provisional application No. 63/216,727, filed on Jun. 30, 2021.

(51) Int. Cl. G16B 30/00; C12Q 1/6874; C12Q 1/6883; C12Q 1/6886; G16B 40/20

(52) U.S. Cl. CPC G16B 30/00; C12Q 1/6874; C12Q 1/6883; C12Q 1/6886; G16B 40/20

(57) ABSTRACT

The present technology relates to methods, computing devices, and systems for identifying somatic mutational signatures (e.g., cancer, aging) from whole genome sequencing (e.g., low coverage WGS) of cell-free DNA (cfDNA) obtained from subjects. Machine learning techniques may be applied to cfDNA mutational profiles, permitting accurate discrimination between cancer patients and healthy individuals or discrimination between different cancer types.

### CROSS-REFERENCE TO RELATED APPLICATIONS

This application claims the benefit of and priority to U.S. Provisional Patent Application No. 63/216,727 filed Jun. 30, 2021, the entire contents of which are incorporated herein by reference.

### TECHNICAL FIELD

The present technology relates to methods, devices, and systems for identifying somatic mutational signatures (e.g., cancer, aging) from whole genome sequencing (e.g., low coverage WGS) of cell-free DNA (cfDNA) obtained from subjects, and the application of machine learning to classify samples based on their SBS mutation profiles.

### BACKGROUND

The following description of the background of the present technology is provided simply as an aid in understanding the present technology and is not admitted to describe or constitute prior art to the present technology.

Mutational signatures accumulate in somatic cells because of endogenous and exogenous processes occurring during an individual's lifetime. Since dividing cells release cell-free DNA (cfDNA) fragments into the circulation, plasma cfDNA may reflect these mutational signatures. Point mutations in plasma whole genome sequencing (WGS) remain largely unexplored due to the limitations of mutation calling from a few sequencing reads.

### SUMMARY OF THE PRESENT TECHNOLOGY

In one aspect, the present disclosure provides a method comprising: performing whole genome sequencing (e.g., low coverage WGS) on cell-free nucleic acids present in a sample comprising whole blood, plasma, and/or serum sample obtained from a subject to identify a plurality of single point mutations; generating a subject sample dataset comprising a patient point mutation profile corresponding to the identified plurality of single point mutations; applying a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and storing, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the methods disclosed herein, the patient point mutation profile comprises a plurality of single base substitution contexts and, a label characterizing each single base substitution context.

In any and all embodiments of the methods disclosed herein the subject sample dataset comprises single nucleotide polymorphisms (SNPs) and the patient point mutation profile comprises at least one mutational signature.

In any and all embodiments of the methods disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the methods disclosed herein, the at least one mutational signature has a mutation count of at least 10, at least 100 or at least 1000.

In any and all embodiments of the methods disclosed herein, the method further comprises removing single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments of the methods disclosed herein, the method further comprises performing principal component analysis (PCA) on the SNP subtracted patient point mutation profile prior to applying the predictive model to the subject sample dataset. Additionally or alternatively, in some embodiments, the method further comprises removing Principal Components with <1% variability prior to applying the predictive model to the subject sample dataset.

In any and all embodiments of the methods disclosed herein, the one or more mutational signatures of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the methods disclosed herein, the one or more mutational signatures of the training set comprises an aging signature.

In any and all embodiments of the methods disclosed herein, the one or more mutational signatures of the training set comprises an APOBEC (apolipoprotein B mRNA editing enzyme, catalytic polypeptide-like) signature.

In any and all embodiments of the methods disclosed herein, the one or more known conditions comprises a cancer.

In any and all embodiments of the methods disclosed herein, the classification comprises a cancer type, or a cancer stage.

In any and all embodiments of the methods disclosed herein, the classification comprises a risk for developing cancer.

In any and all embodiments of the methods disclosed herein, the predictive model employs a gradient boosting machine learning technique.

In any and all embodiments of the methods disclosed herein, the gradient boosting technique comprises an xgboost-based classifier.

In any and all embodiments of the methods disclosed herein, the predictive model employs a decision tree machine learning technique.

In any and all embodiments of the methods disclosed herein, the decision tree machine learning technique comprises a random forest classifier.

In any and all embodiments of the methods disclosed herein, the WGS has a depth between 0.1 and 1.5.

In any and all embodiments of the methods disclosed herein, the WGS has a depth between 0.3 and 1.5.

In any and all embodiments of the methods disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0.

In any and all embodiments of the methods disclosed herein, the WGS has a depth of less than 5.0 or less than 2.0.

In any and all embodiments of the methods disclosed herein, the WGS has a depth of less than 1.0.

In any and all embodiments of the methods disclosed herein, the WGS has a depth of less than 0.3.

In any and all embodiments of the methods disclosed herein, the cohort of study subjects comprises cancer patients, and/or non-cancer patients.

In another aspect, the present disclosure provides a method comprising: (a) generating a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; (b) analyzing a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the methods disclosed herein, the one or more machine learning techniques comprises a gradient boosting learning technique.

In any and all embodiments of the methods disclosed herein, the gradient boosting technique comprises an xgboost-based classifier.

In any and all embodiments of the methods disclosed herein, the one or more machine learning techniques comprises a decision tree learning technique.

In any and all embodiments of the methods disclosed herein, decision tree learning technique comprises a random forest classifier.

In any and all embodiments of the methods disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In another aspect, the present disclosure provides a computing device comprising a processor and a memory comprising instructions executable by the processor to cause the computing device to: perform whole genome sequencing (e.g., low coverage WGS) on cell-free nucleic acids present in a sample comprising whole blood, plasma, and/or serum obtained from a subject to identify a plurality of single point mutations; generate a subject sample dataset comprising a patient point mutation profile corresponding to the identified plurality of single point mutations; apply a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and store, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the devices disclosed herein, the patient point mutation profile comprises a plurality of single base substitution contexts and a label characterizing each single base substitution context.

In any and all embodiments of the devices disclosed herein, the subject sample dataset comprises single nucleotide polymorphisms (SNPs) and the patient point mutation profile comprises at least one mutational signature.

In any and all embodiments of the devices disclosed herein, the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the devices disclosed herein, the at least one mutational signature has a mutation count of at least 10, at least 100, or at least 1000.

In any and all embodiments of the devices disclosed herein, the instructions further cause the computing device to remove single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments of the devices disclosed herein, the instructions further cause the computing device to perform principal component analysis (PCA) on the SNP subtracted patient point mutation profile prior to applying the predictive model to the subject sample dataset. Additionally or alternatively, in some embodiments, Principal Components with <1% variability are removed prior to applying the predictive model to the subject sample dataset.

In any and all embodiments of the devices disclosed herein, the one or more mutational signatures of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the devices disclosed herein, the one or more mutational signatures of the training set comprises an aging signature.

In any and all embodiments of the devices disclosed herein, the one or more mutational signatures of the training set comprises an APOBEC (apolipoprotein B mRNA editing enzyme, catalytic polypeptide-like) signature.

In any and all embodiments of the devices disclosed herein, the one or more known conditions comprises a cancer.

In any and all embodiments of the devices disclosed herein, the classification comprises a cancer type, or a cancer stage.

In any and all embodiments of the devices disclosed herein, the classification comprises a risk for developing cancer.

In any and all embodiments of the devices disclosed herein, the predictive model employs a gradient boosting machine learning technique.

In any and all embodiments of the devices disclosed herein, the gradient boosting technique comprises an xgboost-based classifier.

In any and all embodiments of the devices disclosed herein, the predictive model employs a decision tree machine learning technique.

In any and all embodiments of the devices disclosed herein, the decision tree machine learning technique comprises a random forest classifier.

In any and all embodiments of the devices disclosed herein, the WGS has a depth between 0.1 and 1.5.

In any and all embodiments of the devices disclosed herein, the WGS has a depth between 0.3 and 1.5.

In any and all embodiments of the devices disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the devices disclosed herein, the WGS has a depth between 5.0 and 10.0, between 10.0 and 20.0, or between 20.0 and 30.0.

In any and all embodiments of the devices disclosed herein, the WGS has a depth of less than 5.0 or less than 2.0.

In any and all embodiments of the devices disclosed herein, the WGS has a depth of less than 1.0.

In any and all embodiments of the devices disclosed herein, the WGS has a depth of less than 0.3.

In any and all embodiments of the devices disclosed herein, the cohort of study subjects comprises cancer patients, and/or non-cancer patients.

In another aspect, the present disclosure provides a computing device comprising a processor and a memory comprising instructions executable by the processor to cause the computing device to: (a) generate a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; and (b) analyze a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the devices disclosed herein, the one or more machine learning techniques comprises a gradient boosting learning technique.

In any and all embodiments of the devices disclosed herein, the gradient boosting technique comprises an xgboost-based classifier.

In any and all embodiments of the devices disclosed herein, the one or more machine learning techniques comprises a decision tree learning technique.

In any and all embodiments of the devices disclosed herein, the decision tree learning technique comprises a random forest classifier.

In any and all embodiments of the devices disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In another aspect, the present disclosure provides a computer-readable storage medium comprising instructions executable by a processor to cause of a computing device to cause the computing device to: perform whole genome sequencing (e.g., low coverage WGS) on cell-free nucleic acids present in a sample comprising whole blood, plasma, and/or serum obtained from a subject to identify a plurality of single point mutations; generate a subject sample dataset comprising a patient point mutation profile corresponding to the identified plurality of single point mutations; apply a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and store, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the computer-readable storage medium disclosed herein, the patient point mutation profile comprises a plurality of single base substitution contexts and a label characterizing each single base substitution context.

In any and all embodiments of the computer-readable storage medium disclosed herein, the subject sample dataset comprises single nucleotide polymorphisms (SNPs) and the patient point mutation profile comprises at least one mutational signature.

In any and all embodiments of the computer-readable storage medium disclosed herein, the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the computer-readable storage medium disclosed herein, the at least one mutational signature has a mutation count of at least 10, at least 100, or at least 1000.

In any and all embodiments of the computer-readable storage medium disclosed herein, the instructions further cause the computing device to remove single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments, the instructions further cause the computing device to perform principal component analysis (PCA) on the SNP subtracted patient point mutation profile prior to applying the predictive model to the subject sample dataset. Additionally or alternatively, in some embodiments, the instructions further cause the computing device to remove Principal Components with <1% variability prior to applying the predictive model to the subject sample dataset.

In any and all embodiments of the computer-readable storage medium disclosed herein, the one or more mutational signatures of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the computer-readable storage medium disclosed herein, the one or more mutational signatures of the training set comprises an aging signature.

In any and all embodiments of the computer-readable storage medium disclosed herein, the one or more mutational signatures of the training set comprises an APOBEC (apolipoprotein B mRNA editing enzyme, catalytic polypeptide-like) signature.

In any and all embodiments of the computer-readable storage medium disclosed herein, the one or more known conditions comprises a cancer.

In any and all embodiments of the computer-readable storage medium disclosed herein, the classification comprises a cancer type, or a cancer stage.

In any and all embodiments of the computer-readable storage medium disclosed herein, the classification comprises a risk for developing cancer.

In any and all embodiments of the computer-readable storage medium disclosed herein, the predictive model employs a gradient boosting machine learning technique.

In any and all embodiments of the computer-readable storage medium disclosed herein, the gradient boosting technique comprises an xgboost-based classifier.

In any and all embodiments of the computer-readable storage medium disclosed herein, the predictive model employs a decision tree machine learning technique.

In any and all embodiments of the computer-readable storage medium disclosed herein, the decision tree machine learning technique comprises a random forest classifier.

In any and all embodiments of the computer-readable storage medium disclosed herein, the WGS has a depth between 0.1 and 1.5.

In any and all embodiments of the computer-readable storage medium disclosed herein, the WGS has a depth between 0.3 and 1.5.

In any and all embodiments of the computer-readable storage medium disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the computer-readable storage medium disclosed herein, the WGS has a depth between 5.0 and 10.0, between 10.0 and 20.0, or between 20.0 and 30.0.

In any and all embodiments of the computer-readable storage medium disclosed herein, the WGS has a depth of less than 5.0 or less than 2.0.

In any and all embodiments of the computer-readable storage medium disclosed herein, the WGS has a depth of less than 1.0.

In any and all embodiments of the computer-readable storage medium disclosed herein, the WGS has a depth of less than 0.3.

In any and all embodiments of the computer-readable storage medium disclosed herein, the cohort of study subjects comprises cancer patients, and/or non-cancer patients.

In another aspect, the present disclosure provides a computer-readable storage medium comprising instructions executable by a processor to cause of a computing device to cause the computing device to: (a) generate a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; and (b) analyze a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the computer-readable storage medium disclosed herein, the one or more machine learning techniques comprises a gradient boosting learning technique.

In any and all embodiments of the computer-readable storage medium disclosed herein, the gradient boosting technique comprises an xgboost-based classifier.

In any and all embodiments of the computer-readable storage medium disclosed herein, the one or more machine learning techniques comprises a decision tree learning technique.

In any and all embodiments of the computer-readable storage medium disclosed herein, the decision tree learning technique comprises a random forest classifier.

In any and all embodiments of the computer-readable storage medium disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In one aspect, the present disclosure provides a method for identifying at least one somatic mutational signature in a subject comprising: receiving, by a computing system comprising one or more processors, a whole genome sequencing (WGS) dataset generated by performing, using a next-generation sequencer (NGS), WGS (e.g., low coverage WGS) on cell-free nucleic acids present in a sample comprising whole blood, plasma, and/or serum obtained from a subject; generating, by the computing system, a conditioned dataset by performing a set of operations comprising alignment and GC normalization of sequence reads in the WGS dataset, wherein the WGS dataset is conditioned such that it retains at least a minimum percentage of single nucleotide polymorphisms (SNPs); identifying in the conditioned WGS dataset, by the computing system, single point mutations in the sequence reads in the conditioned WGS dataset based on a comparison of the sequences reads in the conditioned WGS dataset with a reference genome; generating, by the computing system, based on the identified single point mutations, a single base substitutions (SBS) dataset comprising an SBS matrix with a frequency for each mutational variant in a set of SBS variants, wherein the set of SBS variants comprises 96 different contexts, each context corresponding to a unique 3 base pair (bp) combination of a mutated base and two adjacent bases on opposing sides of the mutated base; and applying, by the computing system, a signature fitting technique to the SBS matrix to generate a point mutation profile that is indicative of at least one mutational signature detected in the cell-free nucleic acids present in the sample.

In some embodiments, the method further comprises generating, by the computing system, a correlation score for the point mutation profile for one or more clinical metrics. Examples of the one or more clinical metrics include, but are not limited to, microsatellite instability (MSI), tumor mutation burden (TMB), and mutation count per signature.

Additionally or alternatively, in some embodiments, the method further comprises administering to the subject a treatment based on the generated correlation score. In certain embodiments, the treatment comprises immune checkpoint blockade (ICB) therapy. Examples of ICB therapy include, but are not limited to, a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IB1I308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the sample is a first sample taken prior to a treatment, and the method further comprises: receiving, by the computing system, a second WGS dataset generated by performing WGS on cell-free nucleic acids present in a second sample comprising whole blood, plasma, and/or serum obtained from the subject following the treatment; generating, by the computing system, a second conditioned dataset by performing a set of operations comprising alignment and GC normalization of sequence reads in the second WGS dataset, wherein the second WGS dataset is conditioned such that it retains at least the minimum percentage of SNPs; identifying in the second conditioned dataset, by the computing system, single point mutations in the sequence reads in the second conditioned dataset based on a second comparison of the sequences reads in the second conditioned dataset with the reference genome; generating, by the computing system, based on the identified single point mutations, a second SBS dataset comprising a second SBS matrix with a frequency for each mutational variant in the set of SBS variants; and applying, by the computing system, the signature fitting technique to the second SBS matrix to generate a second point mutation profile that is indicative of at least one mutational signature detected in the cell-free nucleic acids present in the second sample.

In certain embodiments, the method further comprises generating, by the computing system, a second correlation score for the second point mutation profile with respect to at least one of the one or more clinical metrics. Additionally or alternatively, in some embodiments, the method further comprises administering the treatment after the first sample is obtained from the subject. Additionally or alternatively, in certain embodiments, the method further comprises comparing, by the computing system, the first point mutation profile with the second point mutation profile to determine an effect of the treatment on a disease phenotype. In some embodiments, the second point mutation profile lacks a mutational signature identified in the first point mutation profile, and the effect indicates a decrease in a severity or duration of the disease phenotype in the subject.

Additionally or alternatively, in some embodiments, the treatment is a first treatment, and the method further comprises determining, by the computing system, a second treatment based on the effect of the first treatment. In certain embodiments, the method further comprises administering the second treatment for the disease phenotype. Additionally or alternatively, in certain embodiments, the disease phenotype is a cancer, such as colorectal cancer, lung cancer, breast cancer, gastric cancer, pancreatic cancer, bile duct cancer, duodenal cancer, ovarian cancer, uterine cancer, or thyroid cancer.

In any and all embodiments of the methods disclosed herein, the at least one mutational signature has a mutation count of at least 10, at least 100, or at least 1000.

In any and all embodiments of the methods disclosed herein, the minimum percentage of SNPs retained is 25 percent, 50 percent, 75 percent or 95 percent.

In any and all embodiments of the methods disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any of the preceding embodiments of the methods disclosed herein, the at least one mutational signature comprises a smoking signature, an ultraviolet (UV) light exposure signature, a signature derived from mutagenic agents, an aging signature, and/or an APOBEC (apolipoprotein B mRNA editing enzyme, catalytic polypeptide-like) signature.

In any and all embodiments of the methods disclosed herein, the WGS has a depth between 0.1 and 1.5 or between 0.3 and 1.5.

In any and all embodiments of the methods disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0.

In any and all embodiments of the methods disclosed herein, the WGS has a depth of less than 5.0, less than 2.0, less than 1.0, or less than 0.3.

In another aspect, the present disclosure provides a computing system comprising a processor and a memory comprising instructions executable by the processor to cause the computing system to: receive a whole genome sequencing (WGS) dataset generated by performing, using a next-generation sequencer (NGS), WGS on cell-free nucleic acids present in a sample comprising whole blood, plasma, and/or serum obtained from a subject; generate a conditioned dataset by performing a set of operations comprising alignment and GC normalization of sequence reads in the WGS dataset, wherein the WGS dataset is conditioned such that it retains at least a minimum percentage of single nucleotide polymorphisms (SNPs); identify, in the conditioned dataset, single point mutations in the sequence reads in the conditioned WGS dataset based on a comparison of the sequences reads in the conditioned WGS dataset with a reference genome; generate, based on the identified single point mutations, a single base substitutions (SBS) dataset comprising an SBS matrix with a frequency for each mutational variant in a set of SBS variants, wherein the set of SBS variants comprises 96 different contexts, each context corresponding to a unique 3 base pair (bp) combination of a mutated base and two adjacent bases on opposing sides of the mutated base; and apply a signature fitting technique to the SBS matrix to generate a point mutation profile that is indicative of at least one mutational signature detected in the cell-free nucleic acids present in the sample.

In some embodiments, the system is further configured to generate a correlation score for the point mutation profile for one or more clinical metrics. The one or more clinical metrics may comprise microsatellite instability (MSI), tumor mutation burden (TMB), and/or mutation count per signature.

Additionally or alternatively, in some embodiments of the systems disclosed herein, the sample is a first sample taken prior to a treatment, and the system is further configured to: receive a second WGS dataset generated by performing WGS on cell-free nucleic acids present in a second sample comprising whole blood, plasma, and/or serum, wherein the second sample is obtained from the subject following the treatment; generate a second conditioned dataset by performing the set of operations comprising alignment and GC normalization of sequence reads in the second WGS dataset, wherein the second WGS dataset is conditioned such that it retains at least the minimum percentage of SNPs; identify, in the second conditioned dataset, single point mutations in the sequence reads in the second conditioned dataset based on a second comparison of the sequences reads in the second conditioned dataset with the reference genome; generate, based on the identified single point mutations, a second SBS dataset comprising a second SBS matrix with a frequency for each mutational variant in the set of SBS variants; and apply the signature fitting technique to the second SBS matrix to generate a second point mutation profile that is indicative of at least one mutational signature detected in the cell-free nucleic acids present in the second sample.

Additionally or alternatively, in some embodiments, the system is further configured to generate a second correlation score for the second point mutation profile with respect to at least one of the one or more clinical metrics. In certain embodiments, the system is further configured to compare the first point mutation profile with the second point mutation profile to determine an effect of a treatment on a disease phenotype. Additionally or alternatively, in some embodiments, the second point mutation profile lacks a mutational signature identified in the first point mutation profile, and the effect indicates a decrease in a severity or duration of the disease phenotype in the subject. The disease phenotype may be a cancer. Examples of cancer include colorectal cancer, lung cancer, breast cancer, ovarian cancer, uterine cancer, or thyroid cancer. In some embodiments, the treatment is a first treatment, and the system is further configured to determine a second treatment based on the effect of the first treatment.

In any and all embodiments of the systems disclosed herein, the at least one mutational signature has a mutation count of at least 10, at least 100, or at least 1000.

In any and all embodiments of the systems disclosed herein, the minimum percentage of SNPs retained is 25 percent, 50 percent, 75 percent or 95 percent.

In any and all embodiments of the systems disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any of the preceding embodiments of the systems disclosed herein, the at least one mutational signature comprises a smoking signature, an ultraviolet (UV) light exposure signature, a signature derived from mutagenic agents, an aging signature, and/or an APOBEC (apolipoprotein B mRNA editing enzyme, catalytic polypeptide-like) signature.

In any and all embodiments of the systems disclosed herein, the WGS has a depth between 0.1 and 1.5 or between 0.3 and 1.5.

In any and all embodiments of the systems disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0.

In any and all embodiments of the systems disclosed herein, the WGS has a depth of less than 5.0, less than 2.0, less than 1.0, or less than 0.3.

### BRIEF DESCRIPTION OF THE DRAWINGS

FIGS. 1A-IG represent a study outline and characterization of Pointy data, according to various potential embodiments. FIG. 1A: cfDNA libraries were generated from plasma samples from patients with cancer and healthy individuals from two independent cohorts. WGS was performed to 0.3-1.5× coverage. Mutational signatures were extracted from these data, enabling signature profiling and sample classification. FIG. 1B: The number of mutant reads in low-coverage WGS was modelled with different ctDNA fractions and sequencing depths (Supplementary Methods). At low coverage and low ctDNA fractions, true cancer signal would be unlikely to have greater than 1 mutant read at that locus, making conventional mutational calling difficult. Thus, we developed a method called Pointy for cancer detection focused on leveraging this hypothesized low-coverage ctDNA signal. In this model, a TMB of 1 mutation/mb was assumed, and the number of mutant reads per locus was calculated using a binomial distribution. FIG. 1C: The distribution of sequencing coverage is shown for data downsampled to 10M reads (mean coverage=0.28×). Box plots represent median, bottom and upper quartiles, and the whiskers correspond to 1.5×IQR. FIG. 1D: The number of reads at each stage of the Pointy pipeline are shown as box plots. Details on each of the filter steps are outlined in the Methods. FIG. 1E: Boxplots comparing the total number of mutant reads for healthy individuals vs. patients with stage IV CRC, which showed a median 11,786, vs. 9,322 mutations, respectively (p=0.028, two-tailed Wilcoxon test). FIG. 1F: 96-SBS profiles for healthy and CRC plasma WGS samples are shown. They show a cosine similarity of 0.999 (95% CI 0.999-0.999). FIG. 1G: Fragmentation patterns of mutant fragments are shown for both healthy and cancer samples. The mutant fragments in patients with cancer were, on average, 2 bp shorter than mutant reads from healthy samples (mean 146.8 bp vs. 148.9 bp, p=2.2×10⁻¹⁶, Kolmogorov-Smirnov test). Mutations were required to be present in the overlapping region of paired-end sequencing reads from PE100 sequencing, resulting in a shorter size distribution than conventional cfDNA.

FIGS. 2A-2G represent signature profiling in stage IV CRC according to various potential embodiments. FIG. 2A: The number of mutations per mutational signature is shown for aging and MSI signatures in healthy and cancer samples. *, Benjamini-Hochberg (BH)-corrected p<0.05, **, BH-corrected p<0.01. Box plots represent median, bottom and upper quartiles, and the whiskers correspond to 1.5×IQR. FIG. 2B: In silico signature spike-in and assessment of signature fitting efficiency. Signature fitting efficiency was defined as the ratio of the observed vs. expected increase in signature following spiking in known signatures equivalent to sensitivity. Using an averaged SBS mutation profile from healthy control samples, fixed doses of reference signatures were spiked in at 0.1%, 1% and 10% of the total number of background mutations (9,026 mutations total in healthy sample), equating to spiking in 9, 90 and 900 mutations per signature. 50 iterations were used. FIG. 2C: Boxplots of aging and MSI signature contributions in plasma for samples from healthy individuals and patients with CRC. Adjusted p-values (q) are shown (Wilcoxon test). FIG. 2D: The Pearson correlation between tumor fraction and mutation count for each signature is shown for aging and MSI signatures. FIG. 2E: The correlation between tumor mutation burden (TMB) and mutation count per signature is shown for each aging and MSI signature. FIG. 2F: Heatmap of signatures detected in cancer samples (with 95% specificity, Methods). Detected signatures are indicated in red, undetected samples are shown in blue. The microsatellite instability status of each patient is shown, which was determined previously²⁰. The ichorCNA ctDNA fraction for each sample is also shown. FIG. 2G: Classification of plasma samples as either MSI-high/MSS was performed using an xgboost classifier of SBS mutation profiles (Methods). FIG. 2H: Co-spike experiment of SBS1 plus each signature at a ratio of 1:1 (left panel) or 10:1 (right panel). Baseline spike in sensitivity using 10 mutations is shown on the x-axis, and sensitivity with 1:1 or 10:1 SBS1 co-spike is shown on the y-axis. Signatures with zero mutations fitted with nil SBS1 spike-in are not shown. Data point size is proportional to the cosine similarity of that signature to SBS1. Co-spike in of each signature was repeated with 100 iterations with each setting. Signatures with a significant difference in sensitivity, following Benjamini-Hochberg correction, compared to the nil spike-in setting are highlighted in red.

FIGS. 3A-3E represent cancer detection in stage IV CRC according to various potential embodiments. FIG. 3A: SNP-subtracted mutation profiles from 0.3×WGS of plasma from healthy individuals and patients with stage IV CRC were used as input for Principal Component Analysis (PCA). Healthy and cancer samples showed separation in both PC1 and PC2. PC, principal component. Control samples are indicated by ovals. FIG. 3B: The signature contributions to PC1 and PC2 were assessed by fitting signatures to the SBS profile of each PC. SBSn′ indicates SNP-subtracted mutation data fitted to SBSn, where n is an integer. SNP, single nucleotide polymorphism. FIG. 3C: Aging signature contributions (SBS1 and SBS5) were correlated against SBS8′ (SNP-subtracted) to assess for mis-attributed aging mutations in SNP-subtracted data. SBS8′ was significantly correlated with aging signatures (SBS1, p=4.5×10⁻⁶; SBS5, p=0.017, Pearson correlation). FIG. 3D: PC1 and PC2 were correlated against ctDNA fraction determined by ichorCNA. Both PCs showed significant correlation (PC1, p=0.00068; PC2, p=0.0021, Pearson correlation). Samples from healthy individuals are indicated in ovals. FIG. 3E: An xgboost model was used to identify cancer samples vs. healthy using SNP-subtracted mutation profiles. 10-fold cross validation was used to assess classification performance. Receiver Operating Characteristic curves are shown for the following inputs: SNP-retained data (Area Under the Curve, AUC 0.65, 95% CI 0.59-0.71, light blue), SNP-subtracted data (AUC 0.93, 95% CI 0.89-0.96, dark blue), and SNP-subtracted data combined with ichorCNA ctDNA fractions (AUC 0.97, 95% CI 0.94-1.00, red). FIG. 3F: A random forest model was used to classify cancer samples vs. healthy using SNP-subtracted mutation profiles. 10-fold nested cross validation with 500 iterations was used to assess classification performance. A Receiver Operating Characteristic curve is shown for classification of SNP-subtracted data (AUC 0.99, 95% CI 0.98-1.00). SNP, single nucleotide polymorphism.

FIGS. 4A-4G represent signature profiling in stage I-IV non-small cell lung cancer (NSCLC), pancreatic and gastric cancer, according to various potential embodiments. FIG. 4A: Boxplots showing mutation counts per signature (SNPs-retained) from 0.3×WGS of plasma from healthy individuals and patients with stage I-IV NSCLC. Aging, smoking and APOBEC signatures were assessed, guided by known signatures in lung cancer and smoking^{2,17}, which showed significantly greater numbers of SBS1, SBS2, SBS5 and SBS13 in patients with NSCLC compared to healthy individuals (Wilcoxon test). *, BH-corrected p <0.05, **, BH-corrected p<0.01. Box plots represent median, bottom and upper quartiles, and the whiskers correspond to 1.5×IQR. FIG. 4B: Heatmap of plasma signatures detected in patients with stage I-IV NSCLC at 95% specificity per signature (n=21, Methods). Detected signatures within each sample are shown in red, undetected signatures are shown in blue. Disease stage and ctDNA fraction are annotated. FIG. 4C: Pearson correlations between signature contributions and ctDNA fraction (determined by ichorCNA) are shown for patients with stage I-IV NSCLC and healthy individuals. Cancer samples are shown in blue, healthy samples are shown in red. FIG. 4D: Heatmap of plasma signatures detected with 95% specificity in patients with stage I-III pancreatic cancer (n=27). FIG. 4E: Heatmap of signatures detected with 95% specificity in patients with stage I-IV gastric cancer (n=17). FIG. 4F: Mutations in the overlapping region of a paired-end sequencing read can be either discordant or concordant. Discordant mutations are unlikely to be biological signal, and thus may be used to assess sequencing noise. FIG. 4G: For the top 4 SBS contexts of SBS2 (which comprise 97.8% of the signature), the number of concordant and discordant mutations were compared between healthy individuals and patients with NSCLC. Boxplots are shown for concordant mutations (red) and discordant mutations (blue), which showed significantly increased concordant mutations with a constant rate of discordant mutations i.e. sequencing noise. Wilcoxon tests were performed with a BH correction for multiple testing.

FIGS. 5A-5B represent profiling aging signatures in healthy individuals according to various potential embodiments. FIG. 5A: 139 heathy individuals' plasma WGS data (50M reads) from the Cristiano et al.¹³ study were used to study the relationship between aging signatures in plasma and chronological age. As multiple sequencing runs were used, signature contributions were normalized by mean-centering. Signature profiles were assessed with SNPs retained. Correlation between all signatures in healthy individuals was assessed and are shown, which showed a group of signatures that were significantly with SBS1 and SBS5. Only correlations with a significance of p<0.05 are shown in color. FIG. 5B (upper panel): To maximize the signal-to-noise ratio for aging signals, SNP-subtracted signatures were used and were correlated with the chronological age of each healthy individual. SBS8′ showed a small but significant correlation with age following correction for multiple testing (q=0.015, all signatures are shown in FIG. 12). Other signatures were not significantly correlated with age. FIG. 5B (lower panel): In silico size-selection for mutant fragments <150 bp was performed on these data. The SBS8′ (SNP-subtracted) correlation with chronological age was still present after size selection, indicating that aging-associated mutations in healthy individuals are present in short mutant fragments. FIG. 5C: 159 heathy individuals' plasma WGS data (50M reads) from the Cristiano et al.¹⁵ study, sequenced on the same machine, were used to study the relationship between aging signatures in plasma and chronological age. Correlations between signatures were assessed with SNPs-retained, which showed a group of signatures that were significantly correlated with SBS1. Only correlations with a significance of p<0.05 are shown in color. FIG. 5D: SBS1 and SBS1-correlated signatures were tested for their association with aging. Following Benjamini-Hochberg correction, SBS1, SBS30 and SBS33 showed significant association with chronological age (all signatures tested are shown in FIG. 31)

FIGS. 6A-6D: represent cancer detection and classification in stage I-IV NSCLC, pancreatic and gastric cancer, according to various potential embodiments. FIG. 6A: PCA was performed on the SNP-subtracted SBS profiles of patients with stage I-IV NSCLC, pancreatic and gastric cancer using 10M reads (0.3×WGS). PCA showed differences in SBS profile in both PC1 and PC2. FIG. 6B: Classification of all samples as either healthy or cancer was performed using xgboost and 10-fold cross-validation, repeated 10 times, with 10M and 25M reads, which showed AUCs of 0.89 (95% CI 0.86-0.91) and 0.94 (95% CI 0.93-0.95) respectively. FIG. 6C: Using 25M reads, the ROC curves for individual cancer types are shown, which showed AUCs of 0.93 for NSCLC (95% CI 0.92-0.96), 0.93 for pancreatic cancer (95% CI 0.92-0.96) and 0.95 for gastric cancer (95% CI 0.92-0.96). FIG. 6D: With a specificity of 95%, the detection rates per cancer type and stage are shown. Stage=NA denotes healthy individuals. The dashed horizontal line indicates 5% detection (or 95% specificity in healthy individuals).

FIG. 7 represents Pointy pipeline flow diagram according to various potential embodiments. WGS data was trimmed, aligned and GC normalized (Methods). For identification of individual mutational signatures, SNPs were retained in the data, as bulk removal can distort the signature profile (FIG. 10). For classification of samples as either cancer or healthy, SNPs were subtracted to maximize the signal-to-noise ratio (FIG. 10). Individual mutant reads were selected, which were used to generate a matrix of 96-SBS contexts per sample. Signatures were fitted to each 96-SBS matrix for signature profiling. For sample classification, SNP-subtracted data were processed into principal components (PC) using PCA, and PCs used as input for a classification model. For sample classification, xgboost was used, which generated a Pointy score for each sample, ranging from 0 to 1. Pointy scores were used for classification with a threshold of 95% specificity (Methods).

FIGS. 8A-8E represent GC normalization according to various potential embodiments. FIG. 8A: Stage IV colorectal cancer samples from the PGDX cohort were sequenced in two batches on the same sequencer, enabling the study of inter-batch differences. There was no significant difference between the total number of mutations per sample between batches (p=0.48, two-sided Wilcoxon test). Box plots represent median, bottom and upper quartiles, and the whiskers correspond to 1.5× interquartile range (IQR). FIG. 8B: However, PCA of SBS profiles of the same samples showed clustering by sequencing run. FIG. 8C: Before GC-correction, there was a significant difference between sequencing runs in PC2 without p-value correction. Correction for multiple testing was not used in order to maximize sensitivity for possible batch effects. FIG. 8D: The SBS profiles of PC1 and PC2 are shown, which suggests that PC2 is driven by the SBS contexts at the extremes of GC-content. FIG. 8E: Following GC-bias correction, there was no significant difference in any PC. This GC-correction step was therefore incorporated into the pipeline. FIG. 8F: The cosine similarity in SBS profile between healthy samples from each batch (118 vs. 119) was compared with and without GC-correction, using bootstrapping with 100 iterations. GC-corrected samples showed significantly greater cosine similarity (0.999 vs. 0.995, P<2.2×10−16, Wilcoxon test). FIG. 8G: The difference in SBS profiles between each batch (118 vs. 119) is shown for uncorrected (upper) and GC-corrected data (lower). GC correction reduces the magnitude of GC-bias.

FIG. 9A represents relationship between fragment size and point mutations in Pointy data according to various potential embodiments. For both healthy and stage IV CRC samples in the PGDX cohort, the total number of mutations per sample (SNPs included) and the median fragment size were correlated. There was a significant negative correlation between the median insert size and the number of mutant fragments (Pearson r=−0.75, p=2.6×10⁻⁷). This is likely due to mutant fragments released being short in size¹², though shorter fragments will also have a greater overlapping number of base pairs for mutations to be identified in. FIG. 9B: 96-SBS profiles for healthy and CRC plasma WGS samples are shown, which showed a cosine similarity of >0.99. FIG. 9C: Boxplots comparing the total number of mutant reads for healthy individuals vs. patients with stage IV CRC, which showed a median 11,786, vs. 9,322 mutations, respectively (p=0.028, two-tailed Wilcoxon test). This is the raw mutation count (before any background-subtraction).

FIGS. 10A-10D represent comparison of SBS profiles before and after SNP-subtraction according to various potential embodiments. FIG. 10A: Signature fitting with and without SNP-subtraction was performed on both healthy individuals and CRC plasma samples from the PGDX cohort. For each SBS, the median contribution proportion is shown for both SNP-retained and SNP-subtracted data. Following SNP subtraction, SBS1′ (SNP-subtracted) and SBS5′ were no longer assigned mutations, representing a significant decrease (p<1×10⁻¹⁴, two-tailed Wilcoxon test). In contrast, other signatures such as SBS3′, SBS8′ and SBS44′, which were previously not assigned mutations, significantly increased in signature contribution following SNP-subtraction to a median of 8.0% (range 3.4%-18.1%, median p=9.2×10⁻⁷, two-tailed Wilcoxon test). FIG. 10B: The aggregated SBS profile is shown for healthy samples from the PGDX cohort (with SNPs included), compared with the aggregated mutations from the 1000 Genomes database (k1g). The 1000 Genomes database was downloaded, annotated with SBS contexts, and all mutations were combined to generate an aggregated SBS profile. FIG. 10C: Signature fitting to the k1g profile showed that the majority of mutations are attributable to aging signatures (SBS1 and SBS5), and thus subtraction of k1g profile from plasma mutation data may bias signature profiling. The k1g mutation profile was bootstrapped 50 times and signatures were iteratively fitted; box plots represent median, bottom and upper quartiles of the bootstrapped data, and the whiskers correspond to 1.5×IQR. FIG. 10D: SBS profiles are shown for both aggregated healthy and CRC samples from the PGDX cohort following SNP-subtraction using the k1g database. Following SNP-subtraction, cancer samples vs. healthy samples showed a cosine similarity of 0.982 (95% CI 0.982-0.983), compared to 0.999 with SNPs included (95% CI 0.999-0.999, FIG. 1F).

FIGS. 11A-11D represent signature and fragmentation profiling of the DELFI cohort according to various potential embodiments. FIG. 11A: Samples were sequenced across multiple sequencers in the DELFI study. The number of samples per sequencer is shown by cancer type. For this analysis of NSCLC, pancreatic cancer and gastric cancer vs. healthy, all samples were taken from the HWI-D00419 sequencer. FIG. 11B: We compared the signature profiles between the DELFI stage I-IV NSCLC cohort (n=21) and the PGDX stage IV CRC cohort (n=16) to assess the biological correlation of each of the signatures observed. Signature contributions for each sample were background-subtracted using the control samples present in each cohort (Supplementary Methods). For comparisons, Wilcoxon tests were used with a multiplicity correction (BH). Box plots represent median, bottom and upper quartiles, and the whiskers correspond to IQR. FIG. 11C: The correlations between ctDNA fraction and SBS2 contribution in pancreatic cancer and gastric cancer showed no significant correlation, although ctDNA fractions were low in both cohorts, as only 4 out of 27 (14.8%) of patients with pancreatic cancer and 3 out of 15 (20.0%) of patients with gastric cancer had detectable ctDNA using ichorCNA, using a 95% specificity. FIG. 11D: The ratio of fragments below vs. above 150 bp was compared for patients with lung, gastric or pancreatic cancer and healthy individuals. Two-tailed Wilcoxon tests were used to compare median short:long fragment ratios, which showed both significant lengthening (gastric and pancreatic cancer) and shortening (NSCLC) of mutant fragments across cancer type relative to healthy individuals.

FIGS. 12A-12B represent correlation between age and signature contributions in healthy individuals according to various potential embodiments. FIG. 12A: We assessed aging signatures in healthy individuals as we had found aging signatures to be prevalent in both patient and healthy individuals' plasma in the PGDX cohort (FIG. 2A). Therefore, the SBS mutation profiles from 139 healthy individuals from the DELFI cohort were processed similar to previous analyses, except with 50M reads to maximize sensitivity for small differences in signature contribution. Samples were sequenced across three separate sequencing batches. Signatures that were identified to be SBS1- or SBS5-correlated (FIG. 5A) were selected for correlation against chronological age in this analysis. With SNPs included, no signature was significantly correlated with age. Signature contributions for each batch had been normalized by taking the mean SBS contribution for the youngest individuals in each batch (aged 50), then mean differences across the batches in this age group were used to mean-center all data points. Each sequencing batch is shown in a different color. Pearson correlations were used for each SBS. FIG. 12B: Similar to FIG. 12A, SNP-subtracted signatures were correlated against the chronological age of each healthy individual. SBSn′ indicates SBSn with SNP-subtraction. Multiple signatures, including SBS1′ and SBS5′, no longer had any mutations fitted to them, likely due to biased signature fitting caused by SNP subtraction. SBS8′ showed significant correlation with chronological age (corrected p=0.015). FIG. 12C: We sought to assess aging signatures in healthy individuals' plasma after observing aging signatures in patient plasma samples. Therefore, the SBS mutation profiles from 159 healthy individuals sequenced on the same machine from the DELFI cohort were processed as before, except with 50M reads to maximize sensitivity for physiological signatures. Signatures that were identified to be SBS1-correlated (FIG. 5C) were selected for correlation against chronological age in this analysis. Pearson correlations were used for each SBS. Signatures colored in red showed significant correlation with chronological age after correction for multiple testing (q≤0.05, Benjamini-Hochberg method). FIG. 12D: Similar to FIG. 12C, SNP-subtracted signatures were correlated against the chronological age of each healthy individual. SBSn′ indicates SBSn with SNP-subtraction. Multiple signatures, including SBS1′ and SBS5′, no longer had any mutations fitted to them (characterized in FIG. 10), likely due to biased signature fitting caused by SNP subtraction. SBS2′, SBS30′, SBS33′ and SBS46′ showed significant correlation following p-value correction (q<0.03).

FIGS. 13A-13C represent cancer detection and classification in the DELFI cohort according to various potential embodiments. FIG. 13A: For patients with stage IV NSCLC (n=8) in the DELFI cohort, ctDNA detection at baseline using Pointy was associated with poorer progression-free survival (PFS), of 3.9 months vs. median not reached with 18 months' follow-up (HR 6.8, p=0.06, Cox Proportional Hazards model). The small sample size limits the power of this analysis. FIG. 13B: PCA of plasma SBS mutation profiles from patients with stage I-IV NSCLC, pancreatic and gastric cancer samples from the DELFI cohort shows separation of samples in PC1 and PC2. FIG. 13C: Samples from cancer patients from the DELFI cohort were classified to one of their three cancer types using xgboost and 10-fold cross-validation (Methods). All cancer samples, regardless of ctDNA detection status, were included. For each cancer type, the sensitivity and specificity of classification to that cancer type is shown.

FIG. 14A is a block diagram depicting an embodiment of a network environment comprising a client device in communication with server device.

FIG. 14B is a block diagram depicting a cloud computing environment comprising client device in communication with cloud service providers.

FIGS. 14C and 14D are block diagrams depicting embodiments of computing devices useful in connection with the methods and systems described herein.

FIG. 15 depicts a system that includes a computing device and a sample processing system according to various potential embodiments.

FIGS. 16A-16G show input and parameter selection for pointy classification. FIG. 16A: For sample classification, the effect of using raw mutation counts vs. principal components (generated from the same mutation count matrix) as input for xgboost was compared in stage IV MSI CRC plasma samples (raw AUC 0.83 vs. PC-transformed AUC 0.97). The sample classification was performed with SNP-subtracted mutation matrices. Given the increase in the area under the curve (AUC) observed following principal component analysis (PCA), this suggests that dimensionality reduction through PCA enhances the performance of the machine learning classifier. FIG. 16B: In the same stage IV MSI CRC cohort, sample classification using 96 SBS mutation matrices with SNPs either retained or subtracted was compared (SNP-retained AUC 0.88 vs. SNP-subtracted AUC 0.97). In either case, dimensionality reduction using PCA was applied subsequently. These data indicate that SNP removal improves cancer vs. normal sample classification. FIG. 16C: Following PC-transformation of the PGDX MSI plasma data, the number of PCs used for input into the xgboost classifier was varied, and AUCs compared. Above 10 PCs the AUC remained stable. In this data, PCs >10 comprised <1% of the variability in the data, so a threshold of 1% was used to filter PCs. FIG. 16D: The relationship between the number of folds (k) used in k-fold cross validation and the performance of pointy was assessed. For the same stage IV MSI CRC plasma sample data set, the classification performance was measured for k varying between 3 and 15. The AUC ranged from 0.96 to 0.98 with varying values of k. The AUC plateaued above k=10, thus k=10 was used in this study across all cohorts. FIGS. 16E-16G: Comparison of ichor vs. pointy for sample classification (10M reads). Plasma WGS from the DELFI study were used and downsampled to 10M reads per sample. Sample classification was performed using ichor alone, AUC=0.70 (FIG. 16E); pointy alone, AUC=0.86 (FIG. 16F), and pointy and ichor combined, AUC=0.86 (FIG. 16G). Combining ichor and pointy provided no additional detection benefit over pointy alone in this dataset, though there was a non-significant increase in AUC in the PGDX cohort (PGDX pointy alone AUC=0.93 [95% CI 0.89-0.96], pointy and ichor AUC=0.97 [95% CI 0.94-1.00]).

FIG. 17: FASTQ file for a PGDX low-coverage WGS file. Standard FASTQ format is used.

FIG. 18: SAM file for PGDX low-coverage WGS data. The SAM follows standard SAM format.

FIG. 19: Example VCF file. Header not shown. Columns, in order: chr, start, stop, ref, alt, comments. The comment column contains the following standard VCF columns: DP, depth; ADF, alternate depth forward; ADR, alternate depth reverse; AD alternate depth; SGB, strand bias; MQ, mapping quality.

FIG. 20: ANNOVAR-annotated VCF. Header not shown. Columns, in order: chr, start, end, ref, alt, mutation_type, gene_name (and distance to nearest gene).

FIG. 21: GC-bias plot of a representative sample. Picard GCbiasmetrics was used to generate this plot. For each GC % bin, the base quality, the % GC and the normalized coverage is indicated.

FIG. 22: 96-SBS mutation matrix. The value in each cell indicates the number of mutations of each context in each sample.

FIG. 23: SBS matrix (wide). The value in each cell indicates the signature contribution.

FIG. 24: SBS1 contributions for each sample, annotated with MSI status. Value indicates the signature contribution.

FIG. 25: Raw data used for correlation between signature contribution and TMB. SLX_barcode indicates the sample name. tmb indicates TMB in mutations per megabase.

FIG. 26: Correlations between TMB and SBS signature contributions. R indicates correlation coefficient. For each SBS, a linear model between TMB and signature contribution is fitted. A vertical dashed line is plotted at TMB=10mut/mb. The gray shaded area indicates the standard error of the linear model fit.

FIG. 27: AUCs for classification to high/low TMB using SBS signature contribution. A threshold of 10mut/mb was used.

FIGS. 28A-28C: Off-target signature fitting analysis. In silico signature spike-in and assessment of signature fitting specificity. Using a mean-averaged SBS mutation profile from healthy control samples as a background, fixed doses of reference signatures were spiked in with (FIG. 28A) 10, (FIG. 28B) 100 and (FIG. 28C) 1,000 mutations. Signature spiking was performed with 100 iterations. Each panel shows a matrix of signature fitting sensitivity for all signatures spiked into all signatures. Signature fitting sensitivity is defined as the ratio of the observed vs. spiked in mutations. In off-target signatures, a sensitivity of 1 represents entirely off-target fitting.

FIG. 29: Comparison of MSI signature contributions. The contribution of MSI signatures in plasma was compared between patients classified as MSI-H, MSS and healthy individuals from Georgiadis et al. Patients with MSS CRC had similar contributions of MSI signatures as healthy individuals (P>0.05, Wilcoxon test), whereas patients with MSI-H CRC had significantly greater contributions of SBS20 and SBS20 compared to healthy (P<0.008).

FIGS. 30A-30H: Performance comparison of data processing steps and machine learning models. 0.3× plasma WGS data from stage IV CRC patients and healthy individuals were used to test different machine learning models for classification using point mutations and copy number from ichorCNA as input (Methods). (FIG. 30A) First, the classification performance of raw SBS mutation matrix input vs. PCA-transformed input was compared, which favored the latter (raw, AUC=0.83 vs. PCA-transformed AUC=0.94). Therefore, the following methods were tested, each using PCA-transformed input: (FIG. 30B) logistic regression, AUC 0.96 (95% CI 0.91-0.98), (FIG. 30C) random forest, AUC 0.99 (95% CI 0.98-1.00), (FIG. 30D) support vector machine, AUC 0.94 (95% CI 0.89-0.97), and (FIG. 30E) xgboost, AUC 0.96 (95% CI 0.95-0.97). (FIG. 30F) Using a random forest model, sequencing data were iteratively downsampled and classified into cancer vs. healthy, which confirms an AUC of 0.97 (95% CI 0.96-0.98). (FIG. 30G) Classification of samples using SNP-retained data showed a lower AUC of 0.74 (95% CI 0.64-0.87), likely due to SNPs contributing biological noise. (FIG. 30H) Classification of samples using mutations supported by both F and R read of each paired-end read (error-suppressed) vs. either F or R read (non-error-suppressed) showed significant benefit of error-suppression (AUC 0.98 vs. 0.93, P=0.004, Wilcoxon test).

FIGS. 31A-31B: Signature correlations in healthy individuals. (FIG. 31A) 159 heathy individuals' plasma WGS data (50M reads) from the Cristiano et al.³ study were analyzed using Pointy with SNPs retained. The Pearson correlation between all signatures in healthy individuals was assessed. Only correlations with a significance of p>0.05 are shown in color. (FIG. 31B) Signature correlations using data with SNPs-subtracted.

FIGS. 32A-32L. Classification performance using Pointy on the DELFI data set. Cancer detection performance using an RF model in the DELFI data set was assessed across all cancer stages (n=199), using 10-fold nested cross-validation using a random forest model (500 iterations). (FIG. 32A) stage I-IV NSCLC, AUC=0.99 (95% CI 0.99-0.99) (FIG. 32B) stage I-III breast cancer, AUC=0.99 (95% CI 0.99-0.99), (FIG. 32C) stage I-IV CRC, AUC=0.98 (95% CI 0.98-0.98), (FIG. 32D) stage I-IV and X gastric cancer, AUC=0.92 (95% CI 0.92-0.92), (FIG. 32E) stages I, III and IV ovarian cancer, AUC=1.00 (95% CI 1.00-1.00), (FIG. 32F) stage I-III pancreatic cancer, AUC=0.87 (95% CI 0.87-0.88). (FIGS. 32G-J) Detection rates were next assessed by stage: stage I, AUC 0.96 (95% CI 0.96-0.96); stage II, AUC 0.95 (95% CI 0.95-0.95); stage III, AUC 0.97 (95% CI 0.97-0.97); stage IV, AUC 0.97 (95% CI 0.97-0.97). (FIG. 32K) Detection rates by stage and cancer type, using a 95% specificity threshold for detection. Healthy samples are included as stage=NA. (FIG. 32L) PCA of plasma SBS mutation profiles from samples from individuals with CRC, gastric cancer, NSCLC, ovarian cancer or pancreatic cancer, each sequenced on the same sequencer (HWI-D00837). This shows separation of samples in PC1 and PC2, which may allow classification by cancer type.

FIGS. 33A-33B. Batch effects across studies and cancer detection across cohorts (FIG. 33A) PCA of 96-SBS profiles of healthy individuals from each of the datasets used shows evidence of batch effect (PGDX, red; DELFI, blue). (FIG. 33B) To assess the generalizability of Pointy across cohorts, samples from healthy controls and patients with CRC were pooled between the two studies and classification was performed using an RF model with 10-fold nested CV, using 10 iterations.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001)Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Earlier detection of cancer improves the likelihood of eligibility to more effective treatments such as surgery, resulting in a greater chance of survival, reduced morbidity and less expensive treatment⁶. Liquid biopsies are increasingly being utilized for non-invasive cancer detection, prognostication and monitoring³. Current methods for early detection using circulating tumor DNA (ctDNA) detect features of the tumor in plasma, which can be linked to the etiology of the cancer, such as point mutations^{7,8}, copy number alterations^{9,10} or methylation patterns¹¹. Other features in plasma may be related to the biology of cfDNA, such as fragmentation patterns of cfDNA from cancer cells^{12,13}. For early detection, interrogating single base substitution (SBS) signatures that occurred early during cancer development might provide a sensitive approach.

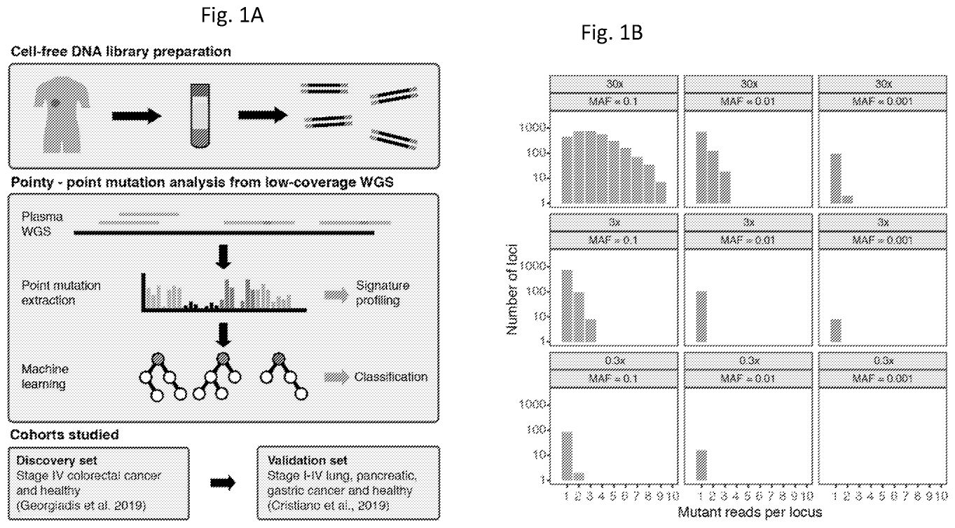

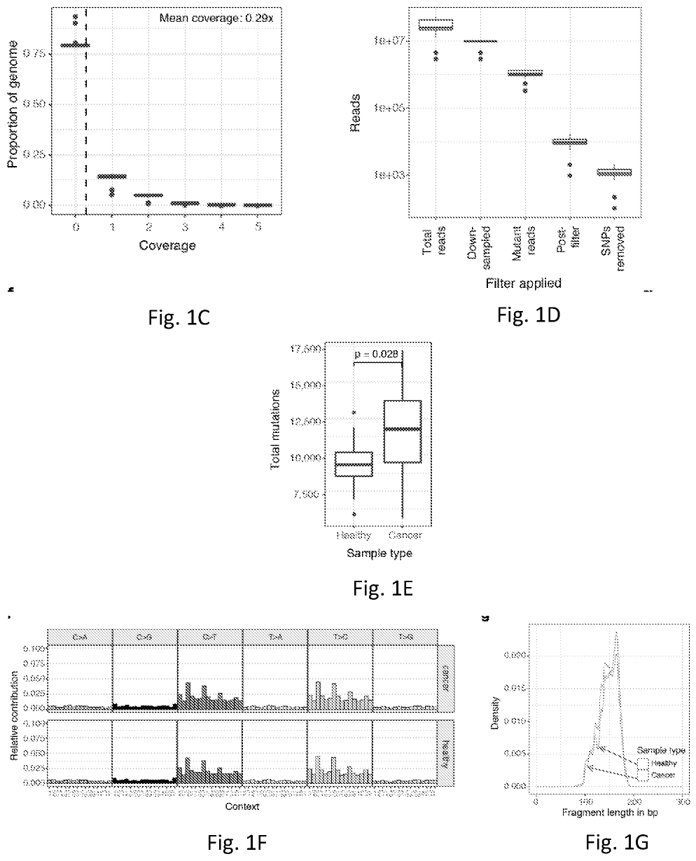

Conventionally, somatic point mutation signature extraction from cancer tissue WGS is performed on confident mutation calls from matched tumor and normal sequencing data at moderate sequencing depth^{2,14}. The present disclosure provides an approach called Pointy to analyze genome-wide mutational signatures from plasma WGS at 0.3-1.5× depth for both signature profiling and sample classification (FIG. 1A). Germline sequencing was not performed to maximize the scalability of this approach, though at the cost of increased biological noise. To mitigate biological and technical noise, for sample classification, we utilized an extreme gradient boosting machine learning algorithm (xgboost)¹⁶.

The present disclosure demonstrates that methods and systems disclosed herein are useful in identifying cancer signatures in patients, and aging signatures in healthy individuals using WGS of plasma cfDNA. For example, by applying machine learning to mutational profiles, patients with stage I-IV cancer were distinguished from healthy individuals with an Area Under the Curve (AUC) of >0.94 in two independent cohorts. The methods of the present technology permit earlier cancer detection, as well as cancer risk based on physiological signatures in plasma. The present disclosure demonstrates that the methods of the present technology showed superior performance with respect to sample classification compared with ctDNA fraction estimates (AUC 0.86 vs. AUC 0.70, respectively).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the terms “amplify” or “amplification” with respect to nucleic acid sequences, refer to methods that increase the representation of a population of nucleic acid sequences in a sample. Nucleic acid amplification methods, such as PCR, isothermal methods, rolling circle methods, etc., are well known to the skilled artisan. Copies of a particular nucleic acid sequence generated in vitro in an amplification reaction are called “amplicons” or “amplification products”.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is colorectal cancer, lung cancer, breast cancer, ovarian cancer, uterine cancer, or thyroid cancer.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Guanine Cytosine (GC) content bias” refers to selection biases related to the sequencing efficiency of genomic regions, whereby read counts depend on sequence features such as GC-content. For instance, GC-rich and GC-poor fragments tend to be under-represented in RNA-Seq, so that, within a lane, read counts are not directly comparable between genes. Additionally, GC-content effects tend to be lane-specific, so that the read counts for a given gene are not directly comparable between lanes. Biases related to length and GC-content confound differential expression (DE) results as well as downstream analyses. As GC-content varies throughout the genome and is often associated with functionality, it may be difficult to infer true expression levels from biased read count measures.

“GC normalization” refers to correction or normalization of the effects of GC content bias on read counts. GC normalization may comprise adjusting for within-lane gene-specific (and possibly lane-specific) effects, e.g., related to gene length or GC-content, and/or effects related to between-lane distributional differences, e.g., sequencing depth.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the terms “individual”, “patient”, or “subject” are used interchangeably and refer to an individual organism, a vertebrate, a mammal, or a human. In a preferred embodiment, the individual, patient or subject is a human.

As used herein, a “mutation” of a gene refers to the presence of a variation within the gene or gene product that affects the expression and/or activity of the gene or gene product as compared to the normal or wild-type gene or gene product. The genetic mutation can result in changes in the quantity, structure, and/or activity of the gene or gene product in a cancer tissue or cancer cell, as compared to its quantity, structure, and/or activity, in a normal or healthy tissue or cell (e.g., a control). For example, a mutation can have an altered nucleotide sequence (e.g., a mutation), amino acid sequence, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the mutations are associated with a phenotype, e.g., a cancerous phenotype (e.g., one or more of cancer risk, oncogenesis, immunogenicity, or responsiveness to treatment). In one embodiment, the mutation is associated with one or more of: a genetic risk factor for cancer, a positive treatment response predictor, a negative treatment response predictor, a positive prognostic factor, a negative prognostic factor, or a diagnostic factor. As used herein, a “missense mutation” refers to a mutation in which a single nucleotide substitution alters the genetic code in a way that produces an amino acid that is different from the usual amino acid at that position. In some embodiments, missense mutations alter one or more functions or physical-chemical properties of the encoded protein.

As used herein, “mutational signatures” refer to characteristic combinations of mutation types arising from specific mutagenesis processes such as DNA replication infidelity, exogenous and endogenous genotoxins exposures, defective DNA repair pathways and DNA enzymatic editing. Examples of mutational signatures include, but are not limited to: endogenous cellular mutations, exogenous carcinogens, Homologous recombination deficiency (HRD), DNA mismatch repair (MMR) deficiency, elevated Cytidine deaminase enzymes, and defective DNA proofreading.

As used herein, a “sample” refers to a substance that is being assayed for the presence of a mutation in a nucleic acid of interest. Processing methods to release or otherwise make available a nucleic acid for detection may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

“Single base substitutions” or “SBS” are defined as a replacement of a single nucleotide base with another single nucleotide base. Exemplary possible substitutions (e.g., labels): C>A, C>G, C>T, T>A, T>C, and T>G. These SBS classes can be further expanded considering the nucleotide context, e.g., considering not only the mutated base, but also the bases immediately 5′ and 3′. In some embodiments, a point mutation profile of a patient may be determined using the conventional 96 SBS mutation type classification or matrices.

As used herein, “SNPs” or “single nucleotide polymorphisms” refer to germline substitutions of a single nucleotide at a specific position in the genome. A SNP segregates in a species' population of organisms.

As used herein, “SNVs” or “single nucleotide variants” are general terms for germline or somatic single nucleotide changes in DNA sequence. In some embodiments, a SNV can be a common SNP or a rare mutation that is caused by cancer.

As used herein, the terms “target gene”, “target sequence” and “target nucleic acid sequence” refer to a specific nucleic acid sequence to be detected and/or quantified in the sample to be analyzed.

### Systems, Devices, and Methods for Modeling

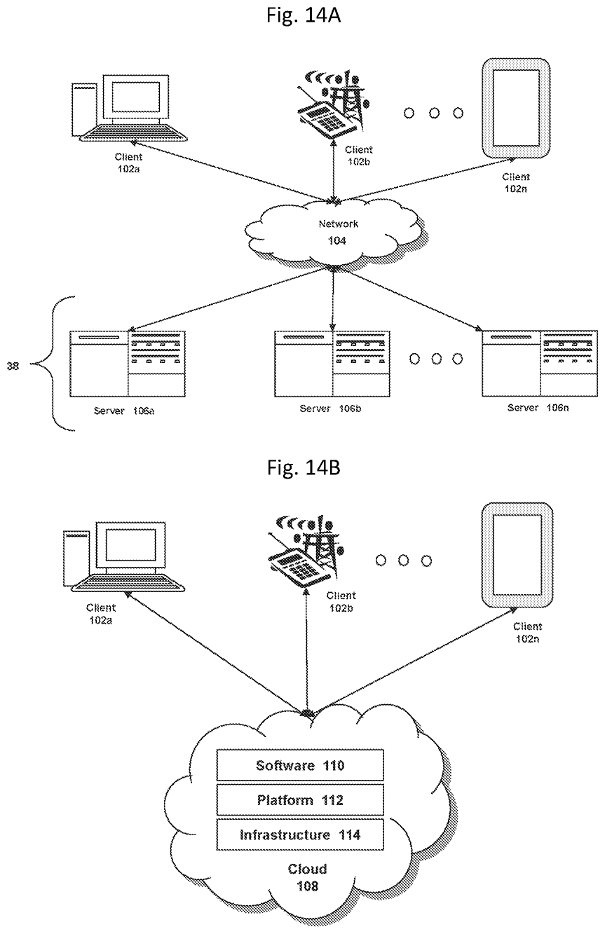

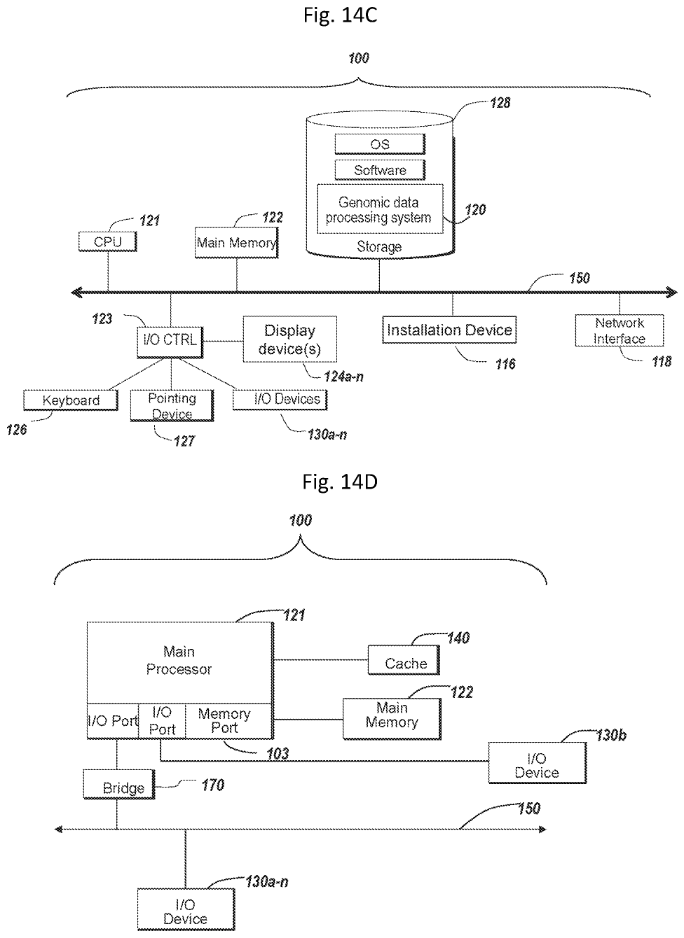

Aspects of the operating environment as well as associated system components (e.g., hardware elements) in connection with various embodiments of the methods and systems described herein will now be discussed. Referring to FIG. 14A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 14A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 14B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 14C and 14D depict block diagrams of a computing device 100 useful for practicing an embodiment of the client 102 or a server 106. As shown in FIGS. 14C and 14D, each computing device 100 includes a central processing unit 121, and a main memory unit 122. As shown in FIG. 14C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 14D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 14C, the processor 121 communicates with main memory 122 via a system bus 150 (described in more detail below). FIG. 14D depicts an embodiment of a computing device 100 in which the processor communicates directly with main memory 122 via a memory port 103. For example, in FIG. 14D the main memory 122 may be DRDRAM.

FIG. 14D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 14D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 14D depicts an embodiment of a computer 100 in which the main processor 121 communicates directly with I/O device 130b or other processors 121′ via HYPERTRANSPORT, RAPIDIO, or INFINIBAND communications technology. FIG. 14D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 14C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 14C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 14B and 14C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

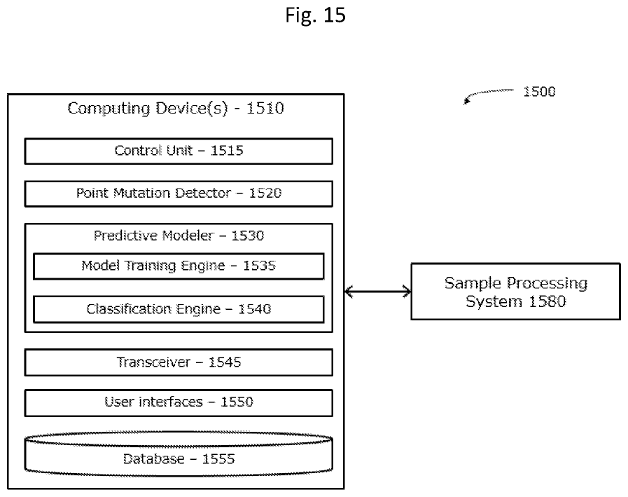

Referring to FIG. 15, in various embodiments, a system 1500 may include a computing device 1510 (or multiple computing devices, co-located or remote to each other) and a sample processing system 1580. In various embodiments, computing device 1510 (or components thereof) may be integrated with the sample processing system 1580 (or components thereof). In various embodiments, the sample processing system 1580 may include, may be, or may employ, in situ hybridization, PCR, Next-generation sequencing, Northern blotting, microarray, dot or slot blots, FISH, electrophoresis, chromatography, and/or mass spectroscopy on such biological sample as blood, plasma, serum, and/or tissue. For example, in certain embodiments, the sample processing system 1580 may be or may include a Next-generation sequencer.

The computing device 1510 (or multiple computing devices) may be used to control, and receive signals acquired via, components of sample processing system 1580. The computing device 1510 may include one or more processors and one or more volatile and non-volatile memories for storing computing code and data that are captured, acquired, recorded, and/or generated. The computing device 1510 may include a control unit 1515 that is configured to exchange control signals with sample processing system 1580, allowing the computing device 1510 to be used to control, for example, processing of samples and/or delivery of data generated and/or acquired through processing of samples. A point mutation detector 1520 may be used, for example, to perform analyses of data captured using sample processing system 1580, and may include, for example, identifying point mutations. A predictive modeler 1530 may be used to implement various machine learning functionality discussed herein. For example, a model training engine 1535 may be used to apply various machine learning techniques (which may comprise, e.g., gradient boosting and/or decision tree techniques) to one or more training datasets (e.g., datasets with genomic data from various cohorts) to train machine learning classifiers for various predictions or other classifications, and a classification engine 1540 may employ a machine learning classifier (e.g., classifiers trained via model training engine 1540) to analyze genomic data (e.g., from one or more patients or other subjects) to make various predictions or other classifications (e.g., cancer type, cancer stage, and/or risk for developing cancer)

A transceiver 1545 allows the computing device 1510 to exchange readings, control commands, and/or other data with sample processing system 1580 (or components thereof). One or more user interfaces 1550 allow the computing device 1510 to receive user inputs (e.g., via a keyboard, touchscreen, microphone, camera, etc.) and provide outputs (e.g., via display screen, audio speakers, etc.). The computing device 1510 may additionally include one or more databases 1555 (stored in, e.g., on or more computer-readable non-volatile memory devices) for storing, for example, data and analyses obtained from or via point mutation detector 1520, predictive modeler 1530 (e.g., model training engine 1535 and/or classification engine 1540), and/or sample processing system 1580. In some implementations, database 1555 (or portions thereof) may alternatively or additionally be part of another computing device that is co-located or remote and in communication with computing device 1510 and/or sample processing system 1580 (or components thereof).

In one aspect, the present disclosure provides a method comprising: performing whole genome sequencing (e.g., low coverage WGS) on cell-free nucleic acids present in a sample comprising whole blood, plasma, and/or serum sample obtained from a subject to identify a plurality of single point mutations; generating a subject sample dataset comprising a patient point mutation profile corresponding to the identified plurality of single point mutations; applying a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and storing, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the methods disclosed herein, the patient point mutation profile comprises a plurality of single base substitution contexts and, a label characterizing each single base substitution context. In any and all embodiments of the methods disclosed herein the subject sample dataset comprises single nucleotide polymorphisms (SNPs) and wherein the patient point mutation profile comprises at least one mutational signature. In any and all embodiments of the methods disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the methods disclosed herein, the at least one mutational signature has a mutation count of at least 10. In any and all embodiments of the methods disclosed herein, the at least one mutational signature has a mutation count of at least 100. In any and all embodiments of the methods disclosed herein, the at least one mutational signature has a mutation count of at least 1000. In any and all embodiments of the methods disclosed herein, the method further comprises removing single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments, the method further comprises performing principal component analysis (PCA) on the SNP subtracted patient point mutation profile prior to applying the predictive model to the subject sample dataset. Additionally or alternatively, in some embodiments, the method further comprises removing Principal Components with <1% variability prior to applying the predictive model to the subject sample dataset.

In any and all embodiments of the methods disclosed herein, the one or more mutational signatures of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the methods disclosed herein, the one or more mutational signatures of the training set comprises an aging signature. In any and all embodiments of the methods disclosed herein, the one or more mutational signatures of the training set comprises an APOBEC (apolipoprotein B mRNA editing enzyme, catalytic polypeptide-like) signature. In any and all embodiments of the methods disclosed herein, the one or more known conditions comprises a cancer. In any and all embodiments of the methods disclosed herein, the classification comprises a cancer type, or a cancer stage. In any and all embodiments of the methods disclosed herein, the classification comprises a risk for developing cancer. In any and all embodiments of the methods disclosed herein, the predictive model employs a gradient boosting machine learning technique. In any and all embodiments of the methods disclosed herein, the gradient boosting technique comprises an xgboost-based classifier. In any and all embodiments of the methods disclosed herein, the predictive model employs a decision tree machine learning technique. In any and all embodiments of the methods disclosed herein, the decision tree machine learning technique comprises a random forest classifier.

In any and all embodiments of the methods disclosed herein, the WGS has a depth between 0.1 and 1.5. In any and all embodiments of the methods disclosed herein, the WGS has a depth between 0.3 and 1.5. In any and all embodiments of the methods disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0. In any and all embodiments of the methods disclosed herein, the WGS has a depth of less than 5.0 or less than 2.0. In any and all embodiments of the methods disclosed herein, the WGS has a depth of less than 1.0. In any and all embodiments of the methods disclosed herein, the WGS has a depth of less than 0.3.

In any and all embodiments of the methods disclosed herein, the cohort of study subjects comprises cancer patients, and/or non-cancer patients.

In another aspect, the present disclosure provides a method comprising: (a) generating a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; (b) analyzing a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the methods disclosed herein, the one or more machine learning techniques comprises a gradient boosting learning technique. In any and all embodiments of the methods disclosed herein, the gradient boosting technique comprises an xgboost-based classifier. In any and all embodiments of the methods disclosed herein, the one or more machine learning techniques comprises a decision tree learning technique. In any and all embodiments of the methods disclosed herein, decision tree learning technique comprises a random forest classifier. In any and all embodiments of the methods disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In another aspect, the present disclosure provides a computing device comprising a processor and a memory comprising instructions executable by the processor to cause the computing device to: perform whole genome sequencing (e.g., low coverage WGS) on cell-free nucleic acids present in a sample comprising whole blood, plasma, and/or serum obtained from a subject to identify a plurality of single point mutations; generate a subject sample dataset comprising a patient point mutation profile corresponding to the identified plurality of single point mutations; apply a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and store, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the devices disclosed herein, the patient point mutation profile comprises a plurality of single base substitution contexts and a label characterizing each single base substitution context. In any and all embodiments of the devices disclosed herein, the subject sample dataset comprises single nucleotide polymorphisms (SNPs) and wherein the patient point mutation profile comprises at least one mutational signature. In any and all embodiments of the devices disclosed herein, the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the devices disclosed herein, the at least one mutational signature has a mutation count of at least 10. In any and all embodiments of the devices disclosed herein, the at least one mutational signature has a mutation count of at least 100. In any and all embodiments of the devices disclosed herein, the at least one mutational signature has a mutation count of at least 1000. In any and all embodiments of the devices disclosed herein, the instructions further cause the computing device to remove single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments, the instructions further cause the computing device to perform principal component analysis (PCA) on the SNP subtracted patient point mutation profile prior to applying the predictive model to the subject sample dataset. Additionally or alternatively, in some embodiments, the instructions further cause the computing device to remove Principal Components with <1% variability prior to applying the predictive model to the subject sample dataset.

In any and all embodiments of the devices disclosed herein, the one or more mutational signatures of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the devices disclosed herein, the one or more mutational signatures of the training set comprises an aging signature. In any and all embodiments of the devices disclosed herein, the one or more mutational signatures of the training set comprises an APOBEC (apolipoprotein B mRNA editing enzyme, catalytic polypeptide-like) signature. In any and all embodiments of the devices disclosed herein, the one or more known conditions comprises a cancer. In any and all embodiments of the devices disclosed herein, the classification comprises a cancer type, or a cancer stage. In any and all embodiments of the devices disclosed herein, the classification comprises a risk for developing cancer. In any and all embodiments of the devices disclosed herein, the predictive model employs a gradient boosting machine learning technique. In any and all embodiments of the devices disclosed herein, the gradient boosting technique comprises an xgboost-based classifier. In any and all embodiments of the devices disclosed herein, the predictive model employs a decision tree machine learning technique. In any and all embodiments of the devices disclosed herein, the decision tree machine learning technique comprises a random forest classifier.

In any and all embodiments of the devices disclosed herein, the WGS has a depth between 0.1 and 1.5. In any and all embodiments of the devices disclosed herein, the WGS has a depth between 0.3 and 1.5. In any and all embodiments of the devices disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the devices disclosed herein, the WGS has a depth between 5.0 and 10.0, between 10.0 and 20.0, or between 20.0 and 30.0. In any and all embodiments of the devices disclosed herein, the WGS has a depth of less than 5.0 or less than 2.0. In any and all embodiments of the devices disclosed herein, the WGS has a depth of less than 1.0. In any and all embodiments of the devices disclosed herein, the WGS has a depth of less than 0.3.

In any and all embodiments of the devices disclosed herein, the cohort of study subjects comprises cancer patients, and/or non-cancer patients.

In another aspect, the present disclosure provides a computing device comprising a processor and a memory comprising instructions executable by the processor to cause the computing device to: (a) generate a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; and (b) analyze a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the devices disclosed herein, the one or more machine learning techniques comprises a gradient boosting learning technique. In any and all embodiments of the devices disclosed herein, the gradient boosting technique comprises an xgboost-based classifier. In any and all embodiments of the devices disclosed herein, the one or more machine learning techniques comprises a decision tree learning technique. In any and all embodiments of the devices disclosed herein, the decision tree learning technique comprises a random forest classifier. In any and all embodiments of the devices disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In another aspect, the present disclosure provides a computer-readable storage medium comprising instructions executable by a processor to cause of a computing device to cause the computing device to: perform whole genome sequencing (e.g., low coverage WGS) on cell-free nucleic acids present in a sample comprising whole blood, plasma, and/or serum obtained from a subject to identify a plurality of single point mutations; generate a subject sample dataset comprising a patient point mutation profile corresponding to the identified plurality of single point mutations; apply a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and store, in one or more data structures, an association between the subject and the one or more classifications. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the computer-readable storage medium disclosed herein, the patient point mutation profile comprises a plurality of single base substitution contexts and a label characterizing each single base substitution context. In any and all embodiments of the computer-readable storage medium disclosed herein, the subject sample dataset comprises single nucleotide polymorphisms (SNPs) and wherein the patient point mutation profile comprises at least one mutational signature. In any and all embodiments of the computer-readable storage medium disclosed herein, the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any and all embodiments of the computer-readable storage medium disclosed herein, the at least one mutational signature has a mutation count of at least 10. In any and all embodiments of the computer-readable storage medium disclosed herein, the at least one mutational signature has a mutation count of at least 100. In any and all embodiments of the computer-readable storage medium disclosed herein, the at least one mutational signature has a mutation count of at least 1000. In any and all embodiments of the computer-readable storage medium disclosed herein, the instructions further cause the computing device to remove single nucleotide polymorphisms (SNPs) from the subject sample dataset prior to applying the predictive model to the subject sample dataset. SNP subtraction permits retention of the cancer signal that is anticipated to be present in somatic SNVs. In certain embodiments, the instructions further cause the computing device to perform principal component analysis (PCA) on the SNP subtracted patient point mutation profile prior to applying the predictive model to the subject sample dataset. Additionally or alternatively, in some embodiments, the instructions further cause the computing device to remove Principal Components with <1% variability prior to applying the predictive model to the subject sample dataset.

In any and all embodiments of the computer-readable storage medium disclosed herein, the one or more mutational signature of the training set comprises a smoking signature, an UV light exposure signature, or a signature derived from mutagenic agents. Examples of mutagenic agents include, but are not limited to, aristolochic acid, tobacco, aflatoxin, temozolomide, benzene, and the like.

In any and all embodiments of the computer-readable storage medium disclosed herein, the one or more mutational signatures of the training set comprises an aging signature. In any and all embodiments of the computer-readable storage medium disclosed herein, the one or more mutational signatures of the training set comprises an APOBEC (apolipoprotein B mRNA editing enzyme, catalytic polypeptide-like) signature. In any and all embodiments of the computer-readable storage medium disclosed herein, the one or more known conditions comprises a cancer. In any and all embodiments of the computer-readable storage medium disclosed herein, the classification comprises a cancer type, or a cancer stage. In any and all embodiments of the computer-readable storage medium disclosed herein, the classification comprises a risk for developing cancer. In any and all embodiments of the computer-readable storage medium disclosed herein, the predictive model employs a gradient boosting machine learning technique. In any and all embodiments of the computer-readable storage medium disclosed herein, the gradient boosting technique comprises an xgboost-based classifier. In any and all embodiments of the computer-readable storage medium disclosed herein, the predictive model employs a decision tree machine learning technique. In any and all embodiments of the computer-readable storage medium disclosed herein, the decision tree machine learning technique comprises a random forest classifier.

In any and all embodiments of the computer-readable storage medium disclosed herein, the WGS has a depth between 0.1 and 1.5. In any and all embodiments of the computer-readable storage medium disclosed herein, the WGS has a depth between 0.3 and 1.5. In any and all embodiments of the computer-readable storage medium disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the computer-readable storage medium disclosed herein, the WGS has a depth between 5.0 and 10.0, between 10.0 and 20.0, or between 20.0 and 30.0. In any and all embodiments of the computer-readable storage medium disclosed herein, the WGS has a depth of less than 5.0 or less than 2.0. In any and all embodiments of the computer-readable storage medium disclosed herein, the WGS has a depth of less than 1.0. In any and all embodiments of the computer-readable storage medium disclosed herein, the WGS has a depth of less than 0.3.

In any and all embodiments of the computer-readable storage medium disclosed herein, the cohort of study subjects comprises cancer patients, and/or non-cancer patients.

In another aspect, the present disclosure provides a computer-readable storage medium comprising instructions executable by a processor to cause of a computing device to cause the computing device to: (a) generate a machine learning classifier that is configured to receive point mutation profiles of patients and output classifications by: (i) providing a whole genome sequencing (e.g., low coverage WGS) library that is obtained by performing WGS of cell-free nucleic acids present in plasma and/or serum samples obtained from a plurality of subjects with a set of one or more predetermined conditions; (ii) generating a training dataset comprising mutational signatures characterizing the one or more predetermined conditions of the plurality of subjects based on the WGS sequence library of (a)(i); and (iii) applying one or more machine learning techniques to the training dataset of (a)(ii) to train the classifier; and (b) analyze a sample dataset for a patient comprising a patient point mutation profile using the trained classifier to obtain a classification for the patient. In some embodiments, the training dataset comprises one or more additional features characterizing the one or more known conditions of the study subjects in the cohort. Examples of such additional features include, but are not limited to, copy number, cfDNA fragmentation, cfDNA fragment end motifs, or cfDNA fragment coordinates.

In any and all embodiments of the computer-readable storage medium disclosed herein, the one or more machine learning techniques comprises a gradient boosting learning technique. In any and all embodiments of the computer-readable storage medium disclosed herein, the gradient boosting technique comprises an xgboost-based classifier. In any and all embodiments of the computer-readable storage medium disclosed herein, the one or more machine learning techniques comprises a decision tree learning technique. In any and all embodiments of the computer-readable storage medium disclosed herein, the decision tree learning technique comprises a random forest classifier. In any and all embodiments of the computer-readable storage medium disclosed herein, the sample dataset is obtained by (i) performing whole genome sequencing (e.g., low coverage WGS) on cell-free nucleic acids present in a plasma and/or serum sample obtained from the patient, to generate a patient sequence library and (ii) generating, based on the patient sequence library, a point mutation profile.

In one aspect, the present disclosure provides a method for identifying at least one somatic mutational signature in a subject comprising: receiving, by a computing system comprising one or more processors, a whole genome sequencing (WGS) dataset generated by performing, using a next-generation sequencer (NGS), WGS (e.g., low coverage WGS) on cell-free nucleic acids present in a sample comprising whole blood, plasma, and/or serum obtained from a subject; generating, by the computing system, a conditioned dataset by performing a set of operations comprising alignment and GC normalization of sequence reads in the WGS dataset, wherein the WGS dataset is conditioned such that it retains at least a minimum percentage of single nucleotide polymorphisms (SNPs); identifying in the conditioned WGS dataset, by the computing system, single point mutations in the sequence reads in the conditioned WGS dataset based on a comparison of the sequences reads in the conditioned WGS dataset with a reference genome; generating, by the computing system, based on the identified single point mutations, a single base substitutions (SBS) dataset comprising an SBS matrix with a frequency for each mutational variant in a set of SBS variants, wherein the set of SBS variants comprises 96 different contexts, each context corresponding to a unique 3 base pair (bp) combination of a mutated base and two adjacent bases on opposing sides of the mutated base; and applying, by the computing system, a signature fitting technique to the SBS matrix to generate a point mutation profile that is indicative of at least one mutational signature detected in the cell-free nucleic acids present in the sample.

In some embodiments, the method further comprises generating, by the computing system, a correlation score for the point mutation profile for one or more clinical metrics. Examples of the one or more clinical metrics include, but are not limited to, microsatellite instability (MSI), tumor mutation burden (TMB), and mutation count per signature.

Additionally or alternatively, in some embodiments, the method further comprises administering to the subject a treatment based on the generated correlation score. In certain embodiments, the treatment comprises immune checkpoint blockade (ICB) therapy. Examples of ICB therapy include, but are not limited to, a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IB1I308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the sample is a first sample taken prior to a treatment, and the method further comprises: receiving, by the computing system, a second WGS dataset generated by performing WGS on cell-free nucleic acids present in a second sample comprising whole blood, plasma, and/or serum obtained from the subject following the treatment; generating, by the computing system, a second conditioned dataset by performing a set of operations comprising alignment and GC normalization of sequence reads in the second WGS dataset, wherein the second WGS dataset is conditioned such that it retains at least the minimum percentage of SNPs; identifying in the second conditioned dataset, by the computing system, single point mutations in the sequence reads in the second conditioned dataset based on a second comparison of the sequences reads in the second conditioned dataset with the reference genome; generating, by the computing system, based on the identified single point mutations, a second SBS dataset comprising a second SBS matrix with a frequency for each mutational variant in the set of SBS variants; and applying, by the computing system, the signature fitting technique to the second SBS matrix to generate a second point mutation profile that is indicative of at least one mutational signature detected in the cell-free nucleic acids present in the second sample.

In certain embodiments, the method further comprises generating, by the computing system, a second correlation score for the second point mutation profile with respect to at least one of the one or more clinical metrics. Additionally or alternatively, in some embodiments, the method further comprises administering the treatment after the first sample is obtained from the subject. Additionally or alternatively, in certain embodiments, the method further comprises comparing, by the computing system, the first point mutation profile with the second point mutation profile to determine an effect of the treatment on a disease phenotype. In some embodiments, the second point mutation profile lacks a mutational signature identified in the first point mutation profile, and the effect indicates a decrease in a severity or duration of the disease phenotype in the subject.

Additionally or alternatively, in some embodiments, the treatment is a first treatment, and the method further comprises determining, by the computing system, a second treatment based on the effect of the first treatment. In certain embodiments, the method further comprises administering the second treatment for the disease phenotype. Additionally or alternatively, in certain embodiments, the disease phenotype is a cancer, such as colorectal cancer, lung cancer, breast cancer, gastric cancer, pancreatic cancer, bile duct cancer, duodenal cancer, ovarian cancer, uterine cancer, or thyroid cancer.

In any and all embodiments of the methods disclosed herein, the at least one mutational signature has a mutation count of at least 10, at least 100, or at least 1000.

In any and all embodiments of the methods disclosed herein, the minimum percentage of SNPs retained is 25 percent, 50 percent, 75 percent or 95 percent.

In any and all embodiments of the methods disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any of the preceding embodiments of the methods disclosed herein, the at least one mutational signature comprises a smoking signature, an ultraviolet (UV) light exposure signature, a signature derived from mutagenic agents, an aging signature, and/or an APOBEC (apolipoprotein B mRNA editing enzyme, catalytic polypeptide-like) signature.

In any and all embodiments of the methods disclosed herein, the WGS has a depth between 0.1 and 1.5 or between 0.3 and 1.5.

In any and all embodiments of the methods disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0.

In any and all embodiments of the methods disclosed herein, the WGS has a depth of less than 5.0, less than 2.0, less than 1.0, or less than 0.3.

In another aspect, the present disclosure provides a computing system comprising a processor and a memory comprising instructions executable by the processor to cause the computing system to: receive a whole genome sequencing (WGS) dataset generated by performing, using a next-generation sequencer (NGS), WGS on cell-free nucleic acids present in a sample comprising whole blood, plasma, and/or serum obtained from a subject; generate a conditioned dataset by performing a set of operations comprising alignment and GC normalization of sequence reads in the WGS dataset, wherein the WGS dataset is conditioned such that it retains at least a minimum percentage of single nucleotide polymorphisms (SNPs); identify, in the conditioned dataset, single point mutations in the sequence reads in the conditioned WGS dataset based on a comparison of the sequences reads in the conditioned WGS dataset with a reference genome; generate, based on the identified single point mutations, a single base substitutions (SBS) dataset comprising an SBS matrix with a frequency for each mutational variant in a set of SBS variants, wherein the set of SBS variants comprises 96 different contexts, each context corresponding to a unique 3 base pair (bp) combination of a mutated base and two adjacent bases on opposing sides of the mutated base; and apply a signature fitting technique to the SBS matrix to generate a point mutation profile that is indicative of at least one mutational signature detected in the cell-free nucleic acids present in the sample.

In some embodiments, the system is further configured to generate a correlation score for the point mutation profile for one or more clinical metrics. The one or more clinical metrics may comprise microsatellite instability (MSI), tumor mutation burden (TMB), and/or mutation count per signature.

Additionally or alternatively, in some embodiments of the systems disclosed herein, the sample is a first sample taken prior to a treatment, and the system is further configured to: receive a second WGS dataset generated by performing WGS on cell-free nucleic acids present in a second sample comprising whole blood, plasma, and/or serum, wherein the second sample is obtained from the subject following the treatment; generate a second conditioned dataset by performing the set of operations comprising alignment and GC normalization of sequence reads in the second WGS dataset, wherein the second WGS dataset is conditioned such that it retains at least the minimum percentage of SNPs; identify, in the second conditioned dataset, single point mutations in the sequence reads in the second conditioned dataset based on a second comparison of the sequences reads in the second conditioned dataset with the reference genome; generate, based on the identified single point mutations, a second SBS dataset comprising a second SBS matrix with a frequency for each mutational variant in the set of SBS variants; and apply the signature fitting technique to the second SBS matrix to generate a second point mutation profile that is indicative of at least one mutational signature detected in the cell-free nucleic acids present in the second sample.

Additionally or alternatively, in some embodiments, the system is further configured to generate a second correlation score for the second point mutation profile with respect to at least one of the one or more clinical metrics. In certain embodiments, the system is further configured to compare the first point mutation profile with the second point mutation profile to determine an effect of a treatment on a disease phenotype. Additionally or alternatively, in some embodiments, the second point mutation profile lacks a mutational signature identified in the first point mutation profile, and the effect indicates a decrease in a severity or duration of the disease phenotype in the subject. The disease phenotype may be a cancer. Examples of cancer include colorectal cancer, lung cancer, breast cancer, ovarian cancer, uterine cancer, or thyroid cancer. In some embodiments, the treatment is a first treatment, and the system is further configured to determine a second treatment based on the effect of the first treatment.

In any and all embodiments of the systems disclosed herein, the at least one mutational signature has a mutation count of at least 10, at least 100, or at least 1000.

In any and all embodiments of the systems disclosed herein, the minimum percentage of SNPs retained is 25 percent, 50 percent, 75 percent or 95 percent.

In any and all embodiments of the systems disclosed herein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, and SBS85, as well rare mutational signatures described in Degasperi et al., (2022) Science 376(6591), which is incorporated herein by reference in its entirety. Examples of rare mutational signatures include but are not limited to SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

In any of the preceding embodiments of the systems disclosed herein, the at least one mutational signature comprises a smoking signature, an ultraviolet (UV) light exposure signature, a signature derived from mutagenic agents, an aging signature, and/or an APOBEC (apolipoprotein B mRNA editing enzyme, catalytic polypeptide-like) signature.

In any and all embodiments of the systems disclosed herein, the WGS has a depth between 0.1 and 1.5 or between 0.3 and 1.5.

In any and all embodiments of the systems disclosed herein, the WGS has a depth greater than 1.0, greater than 2.0, greater than 3.0, greater than 4.0, greater than 5.0, greater than 6.0, greater than 7.0, greater than 8.0, greater than 9.0, greater than 10.0, greater than 20.0, or greater than 30.0. In any and all embodiments of the methods disclosed herein, the WGS has a depth between 5.0 and 10.0, between 10.0 and 20.0, or between 20.0 and 30.0.

In any and all embodiments of the systems disclosed herein, the WGS has a depth of less than 5.0, less than 2.0, less than 1.0, or less than 0.3.

### EXAMPLES

The present technology is further illustrated by the following Examples, which should not be construed as limiting in any way.

### Example 1: Materials and Methods

### Patient and Sample Characteristics

In this study, cfDNA WGS data were analyzed from a total of 82 patients and 39 healthy control individuals across three separate cohorts. For the discovery cohort (PGDX), 16 patients with stage IV CRC and 20 healthy control individuals were recruited, consented and samples were collected as performed as described previously^{20,27}. TMB values for the stage IV CRC cohort were obtained as part of the Georgiadis et al.²⁰ study, which used targeted sequencing on plasma samples. For the validation cohort, 63 patients and 19 healthy control individuals were analyzed from the DELFI¹³ dataset following approval from their Data Access Committee (DAC). For this proof-of-principle study, no blinding or randomization were performed.

For analysis of TMB and MSI in low-coverage WGS, samples were used from 16 patients with stage IV CRC and 20 healthy control individuals who had been previously recruited and consented.

### Plasma Sample Preparation and Sequencing

For patient samples from the PGDX cohort, plasma whole-genome library preparation was performed as described by Georgiadis et al.²⁰ Cell-free DNA (cfDNA) was extracted from plasma using the QIAamp Circulating Nucleic Acid Kit. Libraries were prepared with 5 to 250 ng of cfDNA using the NEBNext DNA Library Prep Kit. Whole-genome libraries were sequenced with a mean of 30M reads using the same sequencing methods as previously described²¹. Experimental methods for the patient samples from the DELFI cohort were previously described¹³.

### Whole Genome Sequencing Data Processing

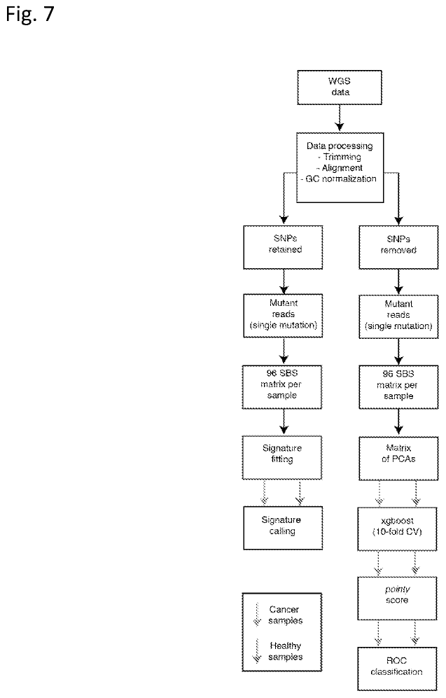

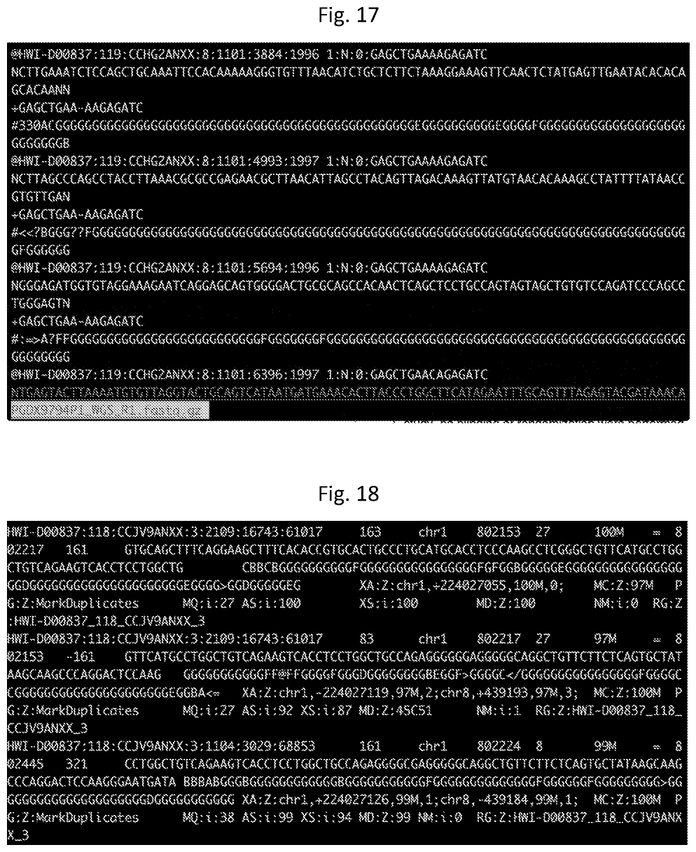

An overview of the pipeline used is shown in FIG. 7. Raw FASTQ files (FIG. 17) were trimmed using trimmomatic (version 0.39)²⁸ in paired-end mode, as follows: all reads were cropped to 100 bp for consistency across datasets (CROP: 100), Illumina sequencing adaptors were removed (ILLUMINACLIP: 2:30:10:2:keepBothReads), leading and trailing 3 bp were trimmed if they were low quality (LEADING: 3, TRAILING: 3), and reads with an average base quality less than 30 were removed (AVGQUAL: 30).

For public datasets, where BAM files were provided, we converted each BAM file to FASTQ using Bedtools (version 2.28.0) bamtofastq prior to running trimmomatic. For all cohorts, sequencer name and batch information were obtained from the read ID from the FASTQ (FIG. 17) using a custom shell script.

Trimmed FASTQ files were aligned to the hg38 genome using BWA (version 0.7.15) mem, sorted and indexed with samtools (version 1.7), and duplicates marked and removed with Picard (version 2.19.0) MarkDuplicates. Indel realignment was performed with GATK (version 3.8). Each BAM was downsampled using Picard (version 2.19.0) DownsampleSam to 10M (PGDX cohort, signature profiling and classification; DELFI cohort signature profiling), or 25M reads (DELFI cohort classification) for cancer detection/classification analyses, or 50M for the study of signatures in healthy individuals. BAM files with <90% of the target number of reads for downsampling were not evaluated (n=2). To maximize the quality of the mapped reads, downsampled BAMs were intersected with UCSC tracks WindowMasker²⁹ and RepeatMasker to remove repeats, then were intersected to retain only regions in the GATK WGS calling regions BED from the GATK hg38 resource bundle. Reads with secondary mapping positions were removed with grep. Reads with a fragment length of zero were removed with awk, as were reads with any supplementary alignments.

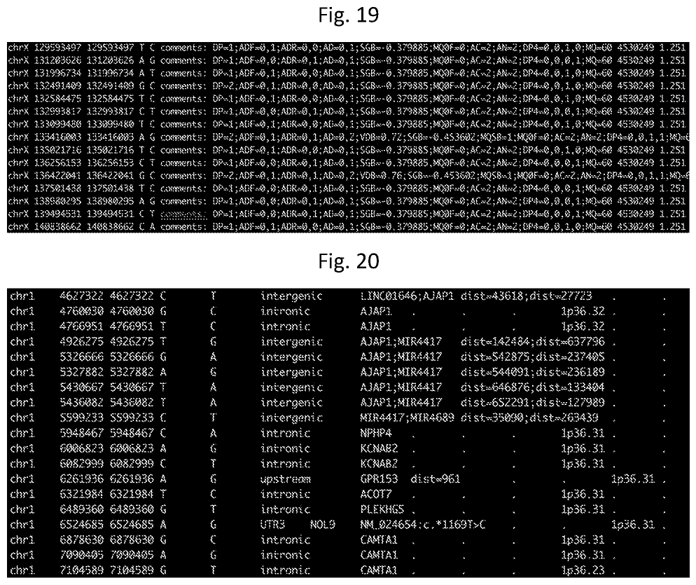

Each BAM file was converted to SAM using samtools (version 1.7) then was filtered using awk to retain mutant reads containing a single point mutation only. Reads from an example SAM file are shown in FIG. 18. Samtools mpileup (version 1.7) was used to identify point mutations, considering only reads with a mapping quality of 60 (-q) and considering mutations only if they had a minimum base quality of 30 (-Q). An example mpileup output is shown in FIG. 19. Indels were removed from the mutation VCF using grep. ANNOVAR (version 2018-04-16) was used to annotate variants using the following databases: refGene, cytoband and dbSNP151. An example ANNOVAR-annotated VCF is shown in FIG. 20. Mutations were annotated as being either concordant, i.e. supported by both R1 and R2 of the same mate pair, or discordant. Annotated and filtered VCFs were read into R (version 3.6) and mutations were annotated with single base substitution contexts using the MutationalPatterns package (version 1.10.0)³⁰.

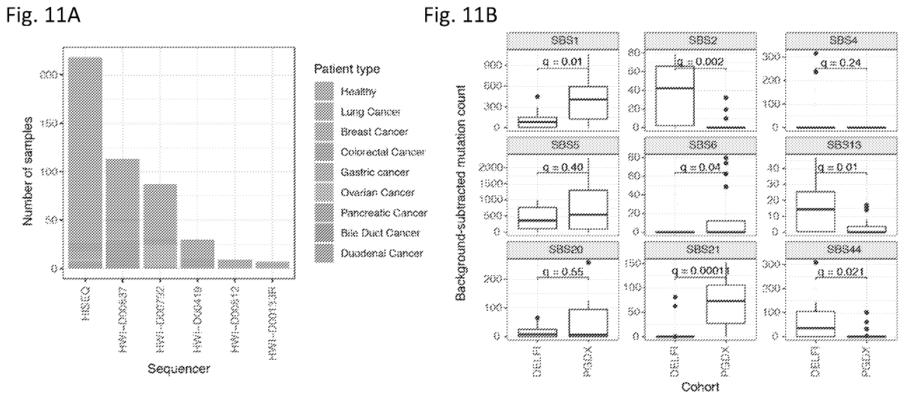

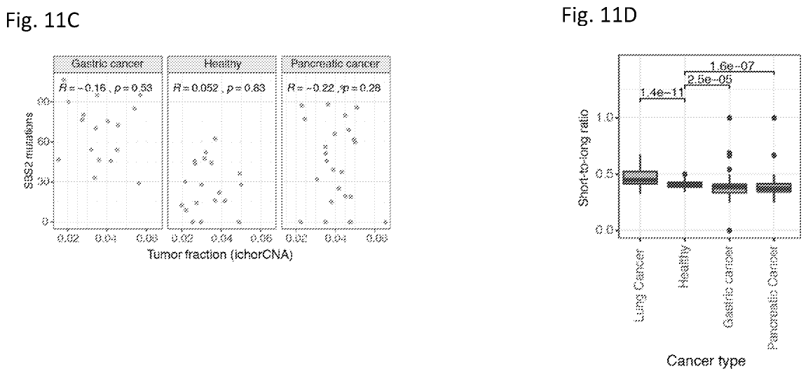

For all samples, the sequencer ID was obtained from the read header in the FASTQ file using a custom shell script (FIG. 11A). To minimize sequencer-specific batch effects on signature profile analysis and sample classification, all downstream analyses were performed by sequencer batch, with patient samples being controlled by healthy individuals on the same sequencer. Two sequencer IDs were excluded due to few samples or only healthy samples being present.

### GC Normalization

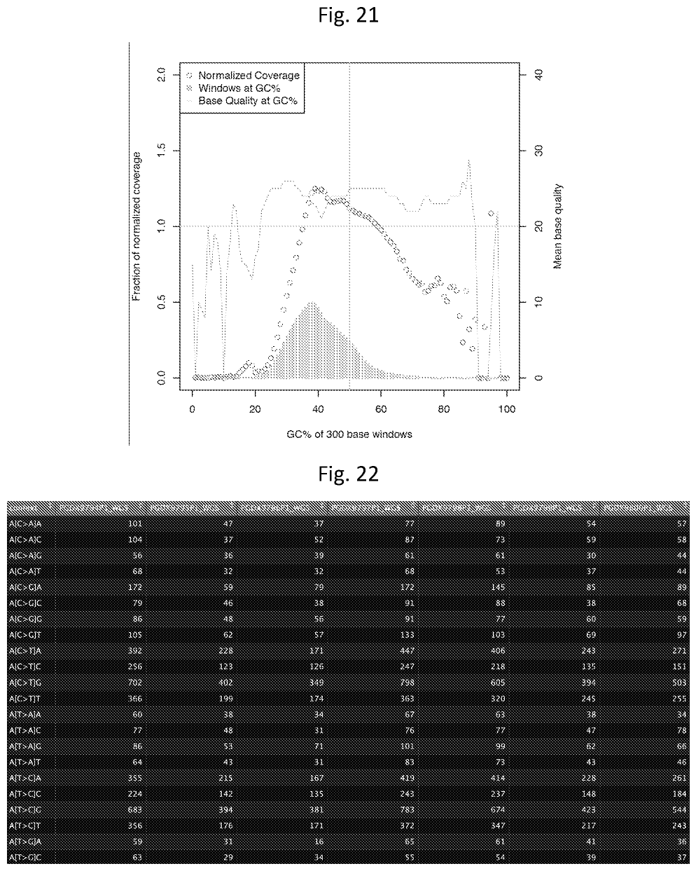

To correct for GC differences between samples within a batch which may influence signature profiles, a GC-bias profile was first determined for each sample. For each sample, we generated a second downsampled BAM file using the same filtering steps, except both mutant and non-mutant reads were retained. The maximum fragment length for consideration for GC bias was set at double the sequencing length (200 bp), since concordant mutations would only be identified in fragments <200 bp using PE100. GC bias metrics were generated using Picard (version 2.19.0) CollectGcBiasMetrics with a WINDOW_SIZE of 300 bp based on previous literature on GC bias in cfDNA³¹. An example GC-bias profile for a sample is shown in FIG. 21.

For all samples from the same cohort that were run on the same sequencer, their GC-bias profiles were aggregated in R, and a generalized additive model (GAM) smoothed fit was used to generate an average profile for the batch using ggplot geom_smooth( ) using method=‘gam’ and formula ‘y˜s(x, bs=“cs”)’.

The averaged GC profile was used to normalize the mutation counts of all samples, based on the GC content of each mutated read as follows: a custom R script was used to annotate all mutations in each sample with their associated GC sequence content, rounded to the nearest 1%. The number of mutations in each GC content % bin was normalized relative to the averaged GC profile belonging to that sequencer, aiming to mitigate differences in GC-bias.

### Mutational Signature Profiling and Detection

For analysis of mutational signatures in patient plasma samples in both cohorts, a 96-SBS mutation profile was generated as described above following filtering, annotation and normalization (example in FIG. 22). For each of the 96 SBS contexts in each sample, the median number of background mutations in that SBS context in control samples was subtracted. Background subtraction was performed relative to control samples sequenced on the same sequencer. This background-subtraction step was performed to maximize signal-to-noise ratio.

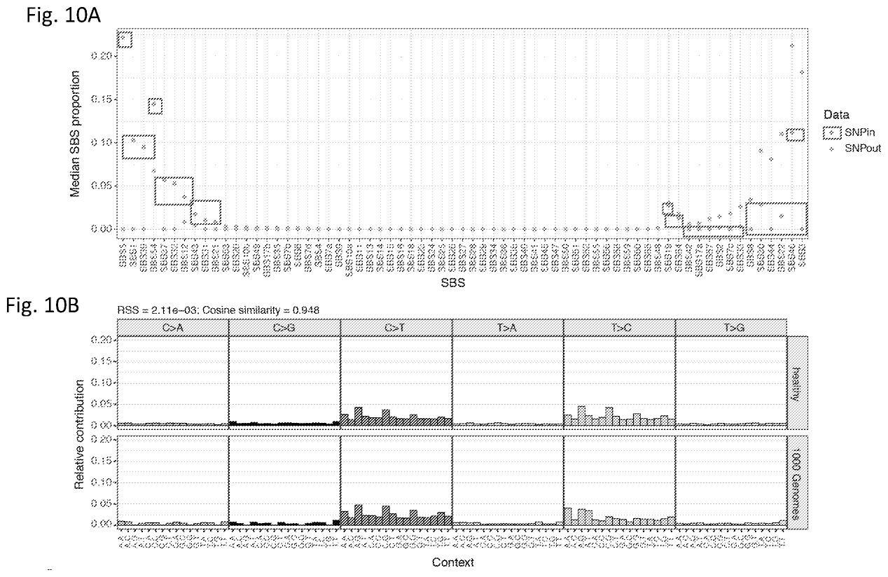

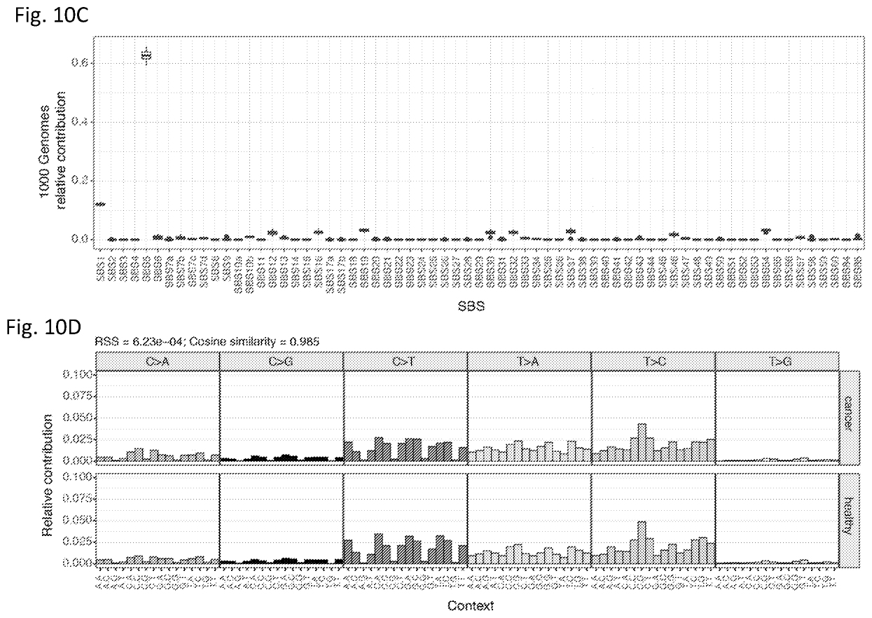

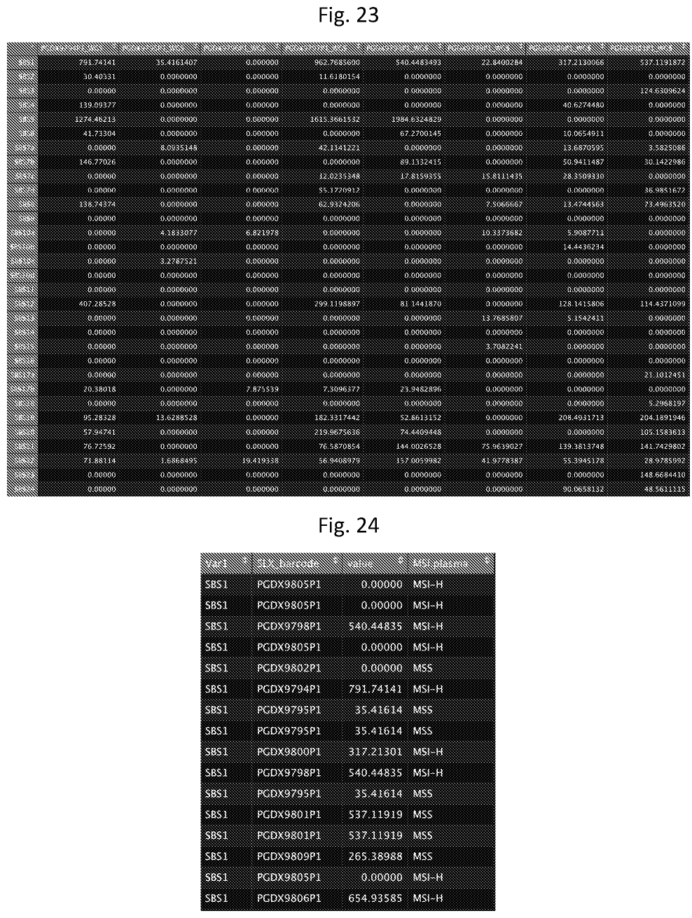

Mutational signatures were fitted using the MutationalPatterns (version 1.10.0)³⁰ fit_to_signature function in R. WGS reference SBS profiles were used². Mutations that had been annotated as SNPs were retained for this analysis, as we showed that removal of SNPs can distort signature fitting processes due to high contributions of aging mutations among SNPs (FIG. 10). For each sample, following signature fitting, a matrix of signature contributions was generated (example in FIG. 23).

To determine whether the signature contribution in an individual sample was significantly above background, we used an empirical threshold for signature detection/calling. For each plasma sample, each signature was considered separately, with a detection threshold set based on the background signal in control samples. The detection threshold for each signature was set using a specificity of 95% in controls, bootstrapped 100 times.

### Sample Classification

For sample classification, SNPs were subtracted to maximize signal:noise. 96-SBS mutation matrices were used as input. For all samples, PCA was used to reduce dimensionality, and Principal Components with <1% variability were removed as a feature selection step. For each sample, a matrix of PCs, annotated with ichorCNA ctDNA fraction, was used as input for the classification model. Samples were classified using controls from the same study and from the same sequencer. For sample classification to either healthy or cancer, we tested multiple classification methods using a nested 10-fold cross-validation method (Vabalas, A. et al., PLoS One 14, e0224365 (2019)), repeated 10 times, using: xgboost, Random Forest (RF), Support Vector Machine (SVM) and Logistic Regression. Nested k-fold cross-validation developed a new model on each training set, with validation on the held-out fold. A nested cross-validation approach has been suggested to be robust to limited sample size (Vabalas, A. et al., PLoS One 14, e0224365 (2019); Varma, S. & Simon, R. BMC Bioinformatics 7, 1-8 (2006)). CreateFolds( ) from the caret package (version 6.0-90) was used to generate balanced folds for each round of cross-validation.

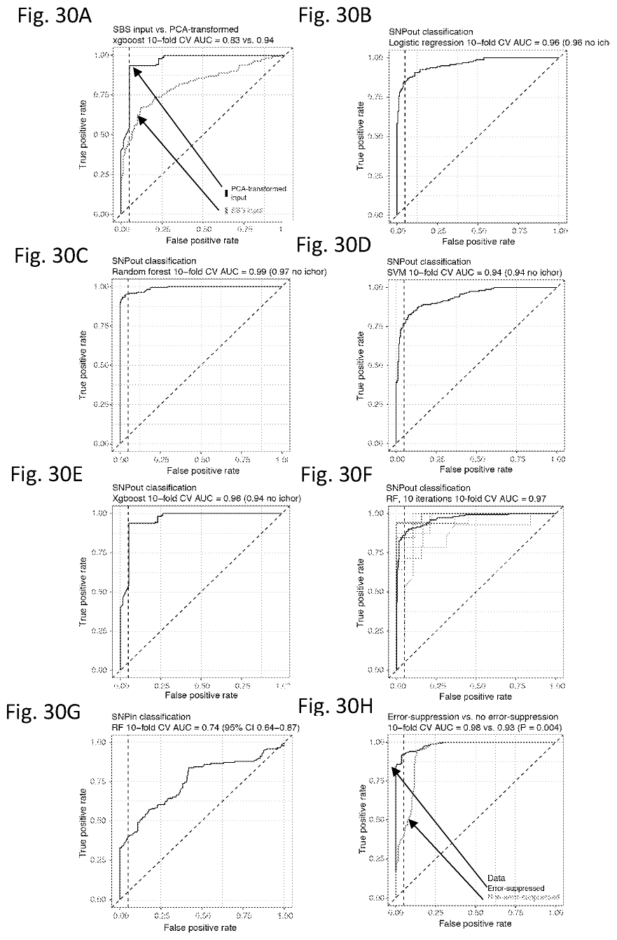

xgboost (v0.90.0.2) was used in R with the default parameters and nrounds=100. randomForest (v4.6-14) was used in R with the default parameters and ntree=500. For SVM, svm( ) from the e1071 package (v1.7.9) was used with default settings. For logistic regression, glm( ) from the stats package (version 4.1.2) was used with default settings. Following each iteration of cross-validation, a Pointy score for each sample was generated, ranging from 0 to 1 (higher represents more likely to be cancer). Classification performance characteristics were determined using the ci.cvAUC function from the cvAUC package (version 1.1.0) in R, using Pointy scores from all iterations as input. Random Forest showed the best performance (FIGS. 30A-30H) and was selected for use subsequently with nested 10-fold cross-validation with 500 iterations. Samples were classified using RF models using this approach for each sequencer within each study. Pointy scores from all iterations from all samples from each study were used as input into ci.cvAUC( ) to generate AUC values by cancer type and stage

A similar approach was used for classification of MSI-H/MSS status of CRC samples, except healthy samples were excluded, and sample labels were either MSI-H or MSS. A threshold of 95% specificity was used for detection of individual samples.

### Classification of Cancer Type

For classification to individual cancer types, healthy samples were excluded, though all cancer samples, regardless of whether ctDNA was detected, were included. Plasma WGS data from the DELFI study were downsampled to 25M reads. For each sample, PCs were extracted from the 96-SBS mutation matrix belonging to each sample (as before), and these were used as input into a random forest classifier. Samples were classified to any of the cancer types present in the dataset using nested 10-fold cross-validation, repeated 10 times. This classifier generates a probability of matching the sample to each class (i.e. cancer type), and the highest scoring class was chosen as the predicted class. In the unlikely event of ties between classes, these were resolved using ties.method=“last”. The classification performance was assessed using a confusion matrix with the confusionMatrix library.

ctDNA Fraction Quantification Using ichorCNA

For all plasma and tumor samples, the ctDNA level (termed as the tumor.fraction) was calculated using ichorCNA (version 0.2.0)⁹, using a window size of 1mb (--window), minimum quality of 20 (--quality), across all autosomes and sex chromosomes (--chromosome), with a maximum copy number of 3 (--maxCN). A panel of normals was not used, but instead, ichorCNA was run across all healthy control samples within each batch. Detection thresholds for ichorCNA were determined in the DELFI cohort using a 95% specificity threshold of ctDNA fractions in healthy individuals in that cohort.

### Data and Materials Availability

Sequence data from patients with CRC from the PGDX cohort will be made available on publication at the European Genome-Phenome Archive, in EGAS00001006377 via a Data Access Committee. DELFI data are publicly available¹³.

### Fragmentation Analysis

To analyze fragment size of Pointy mutations, insert sizes were obtained from the SAM file belonging to each sample. Each raw mutation matrix containing concordant mutations (i.e. present in both F and R mate pairs) was annotated with the insert sizes from the SAM file using a custom R script. Fragments with an insert size >1,000 bp were excluded. A short:long fragment size ratio was calculated for each sample using a threshold of 150 bp.

### Model of Mutant Read Count in WGS.

The number of loci in plasma WGS that may be called by conventional methods was estimated for varying depths of WGS and with varying ctDNA mutant allele fractions (FIG. 1B). In this model, a TMB of 1 mutation/mb was assumed, based on TMB values reported previously³¹. The sequencing coverage at each locus was estimated using a Poisson distribution for each level of sequencing coverage. At each locus, the number of mutant reads per locus was calculated using a binomial distribution based on the sequencing depth and the ctDNA fraction of the sample.

### Quantifying the Accuracy of Signature Fitting.

To assess the accuracy of signature fitting to Pointy data, we performed an in silico signature spiking experiment into a healthy SBS profile, whereby all signatures were each spiked in, with varying numbers of mutations. This allows the assessment of the sensitivity of signature identification using this approach. First, an averaged plasma WGS SBS profile from healthy individuals in the PGDX cohort was generated by taking the median number of mutations per SBS across all samples. Next, fixed doses of each SBS signature were spiked in, of between 10 and 1,000 total mutations per signature. In silico signature spiking was repeated 50 times, and the contribution of each signature was assessed pre- and post-spike.

### Normalization of Signature Contributions Across Batches of Cancer Samples.

To compare signature profiles between samples across different cohorts, for each sample, we subtracted the mean background signal in healthy individuals in its respective cohort. This results in a background-subtracted cancer signal that may be compared across cohorts.

### Normalization of Aging Signature Contributions Across Batches of Healthy Individuals (Limited Cohort Size).

To maximize the power of this analysis, multiple batches of healthy individuals were used to assess the relationship between aging signature contributions and chronological age. Therefore, signature contributions for each batch were normalized by first calculating the mean SBS contributions for the youngest individuals in each batch (aged 50, n=40), to serve as a within-batch background. All data points within each batch were background-subtracted relative to the mean signal in individuals aged 50.

### Mutational Signature Profiling in Healthy Individuals (Large Cohort)

For signature profiling in healthy individuals from the DELFI study, all healthy individuals sequenced on the sequencer named ‘HISEQ’ were analyzed (n=159). Signature fitting was performed as above, except background subtraction was not performed. Signature contributions were correlated against healthy individuals' chronological age from DELFI metadata.

### Example 2: Modeling the Expected Cancer Signal

We first sought to model the expected cancer signal in low-coverage WGS data based on our existing knowledge (Supplementary Methods). At <1× depth, many true mutation loci in Pointy data will have zero coverage. For those loci that are sequenced, somatic and germline mutations would likely be indistinguishable by allele fraction alone (FIG. 1B), precluding the use of per-locus allele fraction-based germline filters^{15,17}. Similarly, conventional mutation calling approaches, which require multiple mutant reads to support the call, may not be used¹⁸. When we modeled high sequencing depths of WGS, dilution of mutant DNA in wild-type cfDNA would still result in many true mutation loci being observed with one mutant read at best when ctDNA levels are low^{15,19} (FIG. 1B). This is due to the long tail of low allele fraction mutations in the tumor being occasionally sampled in plasma. Thus, to interrogate point mutations from low-coverage WGS in this study, we developed methods to extract mutational signatures from individual mutant reads across the genome.

### Example 3: Characterizing and Normalizing Pointy Data

We developed a pipeline to extract point mutations from low-coverage plasma WGS called Pointy (Methods, flowchart in FIG. 7). For the discovery set, we used a cohort of patients with stage IV colorectal cancer (CRC, n=16), many of whom had mismatch repair deficiency (MMR-D) and/or microsatellite instability²⁰ (MSI). Healthy controls from the same cohort were used (n=19). Each library was sequenced to a median of 31.0×10⁶ reads, with a median duplication rate of 0.37%. Data were downsampled to a target of 0.3× (10M paired end reads), which resulted in a median of 10.0×10⁶ reads. A median of 79.3% of genomic positions had zero coverage, and 14% of bases had 1× coverage, equating to a mean coverage of 0.28× (95% CI 0.26-0.29×, FIG. 1C).

In this study, error-suppression by read collapsing of duplicates is limited by the low duplication rate of WGS (<0.5% duplication rate). Instead, we utilized error-suppression filters as follows: minimum base quality (BQ) threshold of 30, mean BQ threshold of 30, requiring mutations to be present in both read 1 (R1) and read 2 (R2), and mapping quality (MQ) threshold of 60. After applying these filters, a mean of 9,886 mutations per sample were retained (95% CI 8,782-10,990, FIG. 1D). Of these high-quality mutations, a median of 87.8% of the mutations per sample were marked as single nucleotide polymorphisms (SNP) using dbSNP. All mutations, including those flagged as possible SNPs, were included for exploratory analysis.

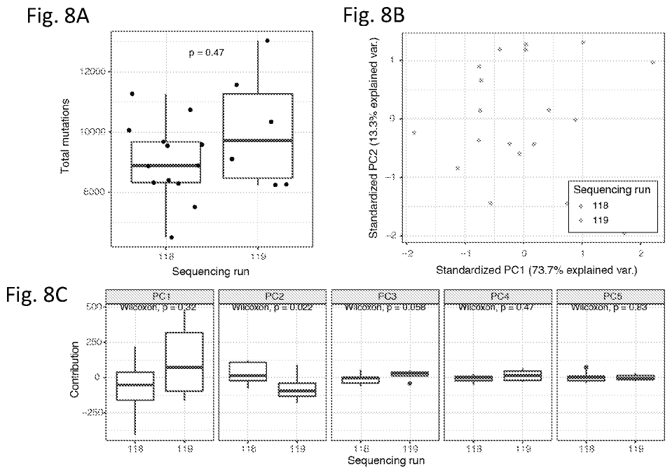

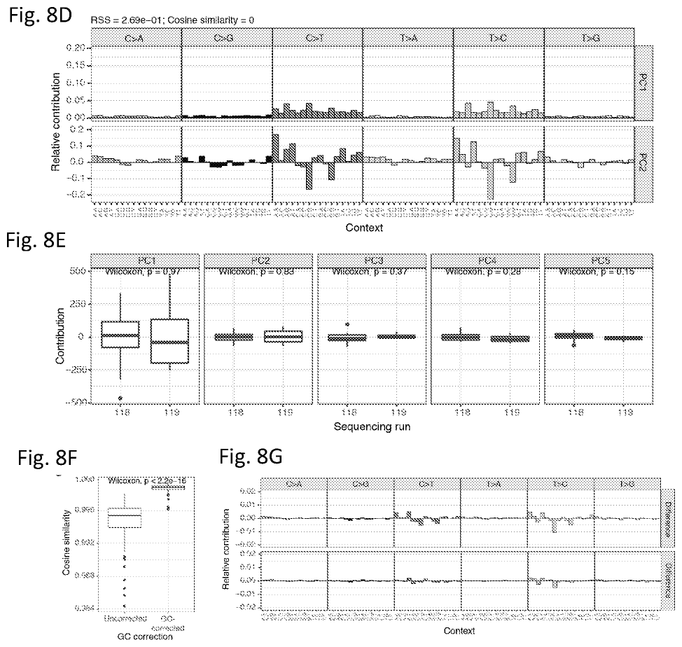

The samples from the discovery cohort were sequenced in two batches from the same sequencing instrument, so we explored data from healthy individuals for batch effects. In healthy samples, there was no significant difference in the mean number of mutations between batches (9,049 vs. 10,089, p=0.47, two-tailed Wilcoxon test, FIG. 8A). However, Principal Component Analysis (PCA) of SBS profiles revealed evidence of batch effect difference in mutation profile, which may arise from differences in GC-bias between sequencing runs. Principal Component Analysis (PCA) of SBS profiles revealed a small but significant difference in the mean contribution of PC2 per sample (unadjusted p=0.022, two-tailed Wilcoxon test, FIGS. 8B, 8C). We observed that the largest contributors to PC2 were contexts at the extremes of GC content (FIG. 8D). Therefore, the GC bias for each sample was determined, as was the average GC profile of the sequencing batch, which were combined to normalize the SBS profile of each sample (Methods). This approach is analogous to GC-correction methods used to correct whole genome copy-number^{9,21} or fragmentation profiles¹³. After GC-correction, there were no significant differences in any PC between the two sequencing runs (unadjusted p>0.05, two-tailed Wilcoxon test, FIG. 8E).

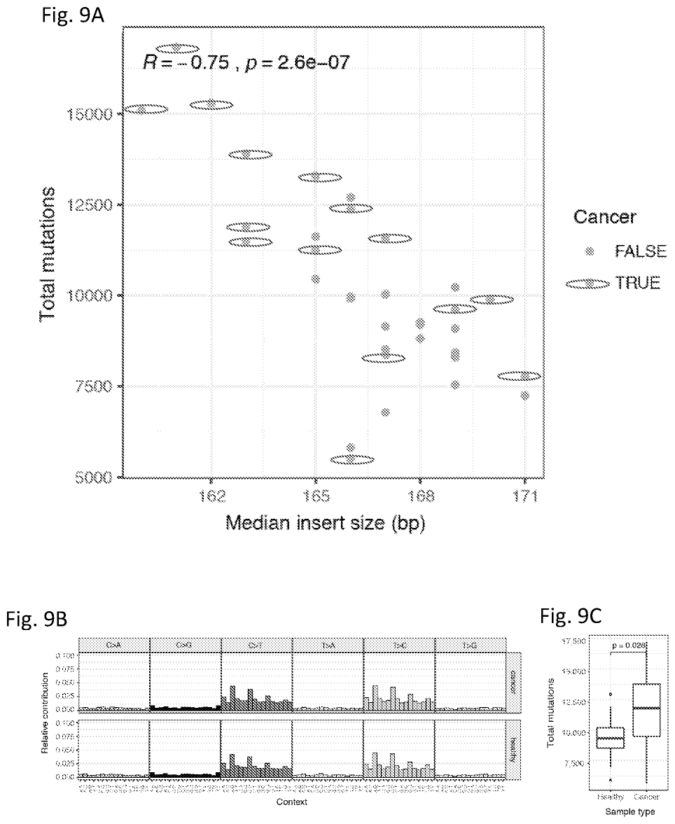

In FIG. 8F, we show high cosine similarities between sample even without GC-correction, though this increased significantly following GC-correction (0.995 vs 0.999, P<2.2×10⁻¹⁶, Wilcoxon test). The difference in SBS profiles between batches with and without GC-correction is shown in FIG. 8G. Following GC-bias normalization, cancer patient plasma samples and healthy controls showed SBS mutation profiles that had a cosine similarity of 0.999 (95% CI 0.999-0.999, FIG. 9B), although this included SNPs.

Cancer patient plasma samples had significantly more point-mutated reads compared to healthy controls (median 11,786, vs. 9,322, p=0.028, two-tailed Wilcoxon test, FIG. 1E). While the absolute number of mutations differed between cases and controls, when aggregated and considered by SBS context, their SBS profiles showed a cosine similarity of 0.999 (95% CI 0.999-0.999, FIG. 1F).

The fragment sizes of mutant reads were also assessed, which showed mutant reads in cancer samples were, on average, 2 bp shorter than mutant reads in healthy samples (mean 146.8 bp vs. 148.9 bp, p=2.2×10⁻¹⁶, Kolmogorov-Smirnov test, FIG. 1G), consistent with previous studies showing shortening of ctDNA relative to cfDNA^{12,22}. Accordingly, there was a negative correlation between the fragment size of a given sample and the number of mutant reads (Pearson r=−0.75, p=2.6×10⁻⁷, FIG. 9A).

### Example 4: Mutational Signatures can be Detected in Plasma

To explore the processes contributing to the mutation profile of each sample, we fitted the data to a database of known mutational signatures² after background subtraction (Methods). For each sample, for each SBS context, the median number of mutations in controls was subtracted. Sequencing artefact signatures were included in the database to minimize misattribution of mutations to biologically relevant signatures.

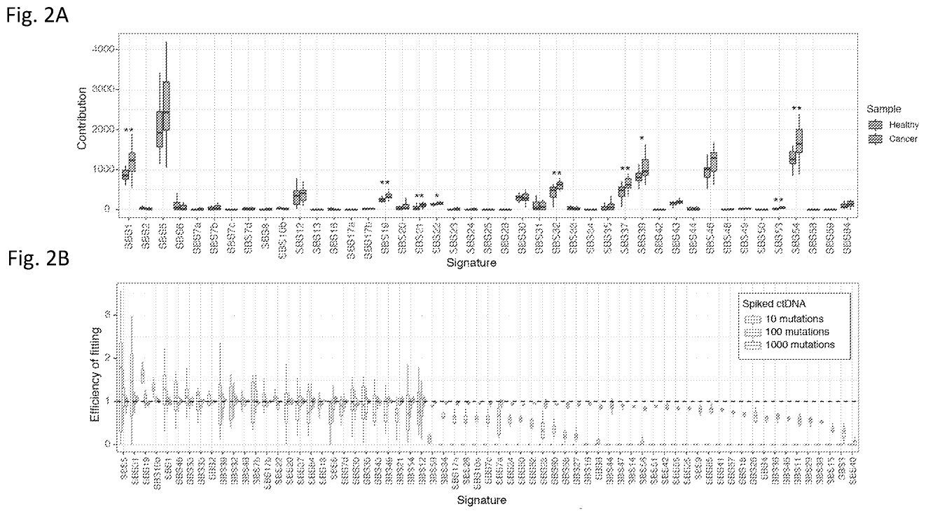

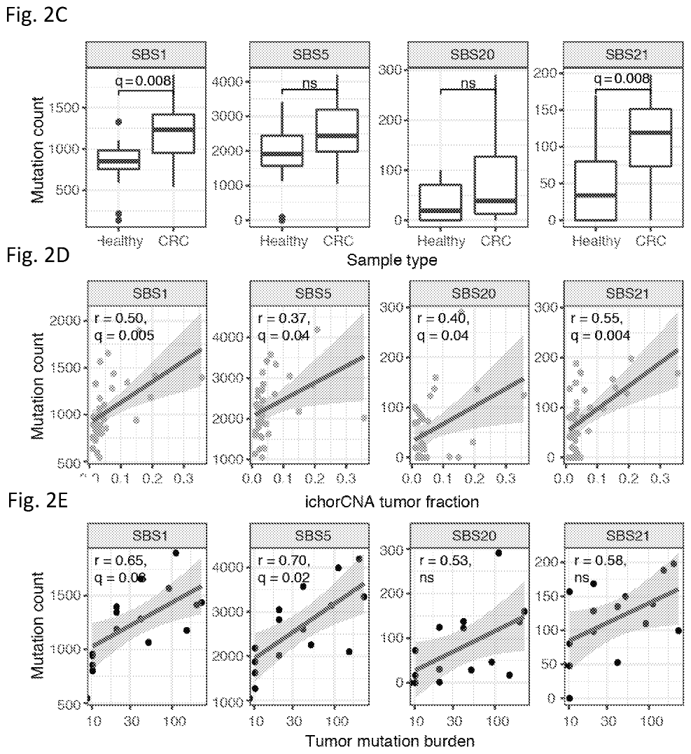

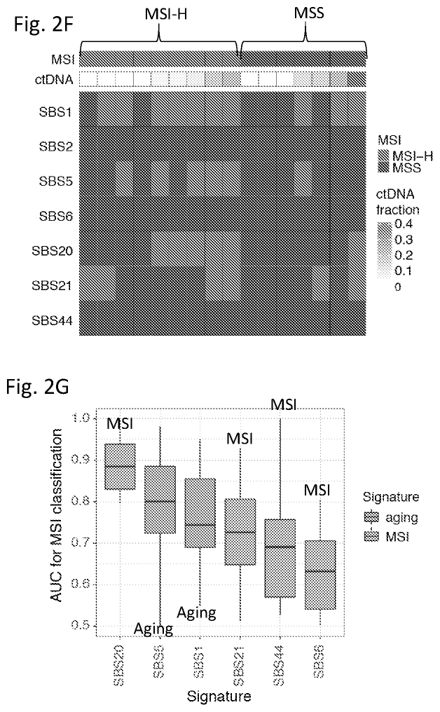

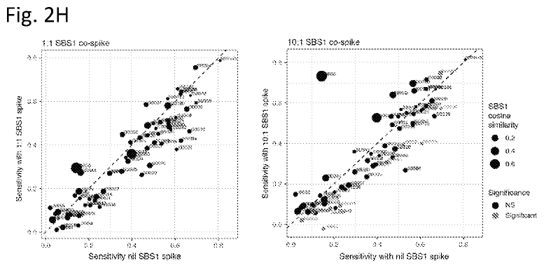

In healthy samples, the largest contributors to plasma Pointy signatures were aging mutations (SBS1 and SBS5), which comprised a median of 888 (9.5%) and 1,934 (21.0%) mutations, respectively (FIG. 2A). Despite the filters applied, SNP contamination (SBS54) was attributed 1,260 (14.0%) mutations, and sequencing artefact (SBS46) had 1,003 (11.0%). Compared to healthy individuals, CRC patient plasma showed significantly greater contributions of SBS1 (Benjamini-Hochberg (BH) adjusted p=0.008, one-sided Wilcoxon test), indicating aging. Also, SBS21 was significantly increased in patients with CRC (adjusted p=0.008, one-sided Wilcoxon test), consistent with previously detected microsatellite instability (MSI) in these patients²⁰, associated with mismatch repair (MMR) deficiency.

To assess the accuracy and sensitivity of signature fitting, an averaged plasma WGS SBS profile from control individuals was generated, and fixed doses of each SBS signature was spiked in between 10 and 1,000 total mutations per signature, repeated 50 times (Supplementary Methods). The contribution of each signature was assessed pre- and post-spike. When 10 mutations per signature were spiked in, 25 out of 67 signatures (37.3%) showed an increase of ≥9 mutations, i.e. ≥90% efficiency of fitting, which included SBS1, SBS2, SBS5, SBS20 and SBS21 (FIG. 2B). This increased to 42 out of 67 signatures (63.0%) when 1,000 mutations per signature were spiked in. Thus, while Pointy may detect aging, APOBEC and MSI signatures, other signatures may be falsely negative due to the signature fitting approach used.

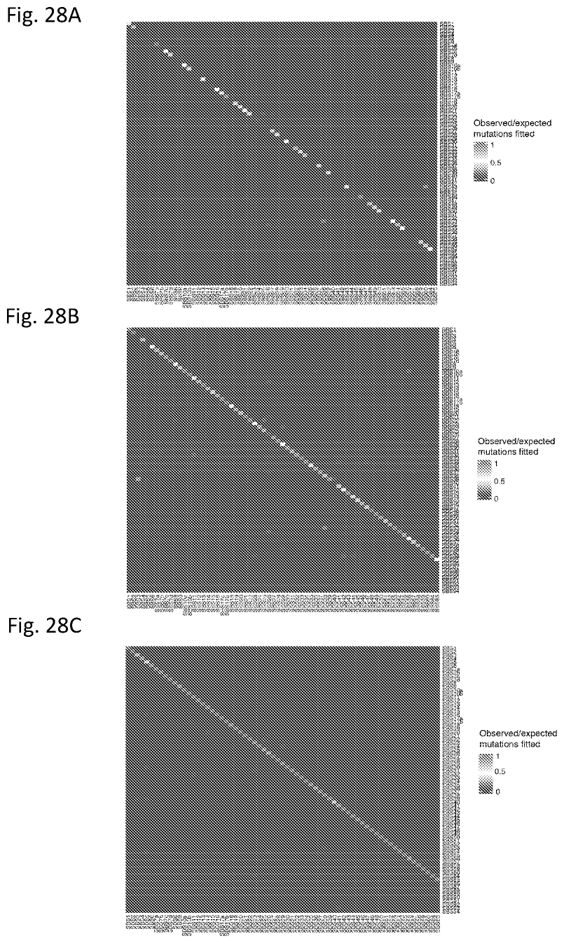

To assess the performance of signature recovery in the setting of multiple signatures, we iteratively spiked in signatures and simultaneously spiked in SBS1 at a ratio of 1:1 or 10:1 (FIG. 2H). At a 1:1 ratio of spike-in of both signatures, there was no impact on signature fitting. However, when 10× more SBS1 mutations were spiked in compared to the signature of interest, the rate of on-target signature fitting was reduced in multiple signatures (Benjamini-Hochberg corrected p<0.05), especially signatures with low cosine similarity to SBS1 (linear regression p=1.5×10⁻⁹). Signatures with similarity to SBS1 gained mutations directly from SBS1 (q>0.05), whereas signatures with low similarity to SBS1 lost mutations, likely to other signatures gaining from SBS1. We show the extent of false positive signature fitting in the context of a singly spiked signature in FIGS. 28A-28C, where the proportion of mutations that were mis-attributed ranged from 1.7% with 10 mutations spiked, to 0.1% with 1,000 mutations spiked.

As aging and MSI signatures had significantly higher contributions in the plasma of patients with CRC in the 10M downsampled data and remained significant when iteratively downsampled 50 times (FIGS. 2A, 2C), we tested whether plasma signature contributions correlated with both ctDNA fraction and tumor mutation burden (TMB). ctDNA fraction was determined by ichorCNA⁹ and tumor mutation burden was determined by targeted panel sequencing of plasma²¹. Multiple aging and MSI-associated signatures showed significant correlation with ctDNA fraction, including SBS1, SBS5, SBS20 and SBS21 (adjusted p<0.05, FIG. 2D). As aging signatures are known to be prevalent in CRC tumors², these data suggest that as ctDNA fraction increases, this increases the likelihood of sequencing aging mutations in plasma using WGS. Furthermore, SBS1 and SBS5 were significantly correlated with TMB (adjusted p<0.05, FIG. 2E), but SBS20 and SBS21 were not. Given that ubiquitous intratumor mutations are more readily detected in plasma²³, we suggest that this bias is also present in plasma signature profiling, which may influence the above results.

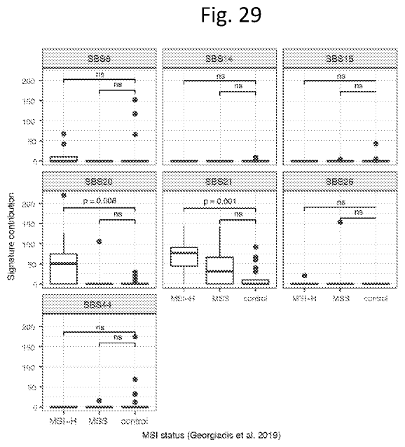

To detect individual signatures per sample, as opposed to comparing the aggregate signature across each group, signature detection was performed (Methods). For each cancer sample, signature detection was performed using the healthy samples as a panel of normals, with a threshold of 95% specificity for each signature. Aging signatures were detected in 10 out of 16 (62.5%) patients, and MSI signatures in 9 out of 16 (56.3%, FIG. 2F). Patients with MSI-H tumors had significantly greater SBS20 and SBS21 contributions than controls, whereas patients with MSS tumors were non-significantly different (FIG. 29).

Classification to MSS/MSI-high using Pointy. Aging and MSI signature contributions were tested for their ability to classify samples as either MSI-H vs. MSS. Signatures known to be associated with MSI were selected, analyzed: SBS6, SBS14, SBS15, SBS20, SBS21, SBS26, SBS44. Aging signatures (SBS1 and SBS5) were also included for comparison. For each signature, a matrix was generated with the signature contribution in each sample (transposed from FIG. 23), annotated with the MSI status of that sample (example in FIG. 24). To test the ability of the above signatures to classify for MSI status, Receiver Operating Characteristic analysis was performed on data that were bootstrapped 25 or 50 times. SBS20 showed the highest median AUC of 0.88 (FIG. 2G), and aging signatures also showed moderate ability to classify MSI (median AUC 0.78). There was no significant difference in the ctDNA tumor fraction between patients with MSI vs. MSS (p=0.56, two-sided Wilcoxon test), suggesting that classification is driven by differences in mutation rate rather than ctDNA fraction. The median threshold for SBS20-based MSI classification was 29.6 mutations (IQR 20.4-56.7).

SNP subtraction and signature fitting. Signature fitting was repeated on the same Pointy data with SNP subtraction. Following SNP subtraction, SBS1′ (SBS1′=SBS1 with SNP subtraction) and SBS5′ were assigned a median of zero mutations (0%) each, representing a significant decrease relative to their SNP-retained counterparts (p<1×10⁻¹⁴, two-tailed Wilcoxon test, FIG. 10A). In contrast, other signatures such as SBS3′ (SNP-subtracted), SBS8′ and SBS44′, which were previously not assigned mutations, significantly increased in signature contribution following SNP-subtraction to a median of 8.0% (range 3.4%-18.1%, median p=9.2×10⁻⁷, two-tailed Wilcoxon test).

We hypothesized that the SNP database contained aging mutations, which were being subtracted from Pointy data. The SBS profile of the aggregated mutations from the 1000 Genomes database, which contains high-quality SNPs²⁴, was compared against that of healthy individuals (FIG. 10B). We confirmed that the largest signature contributions were from SBS1 (12.1%) and SBS5 (63.2%, FIG. 10C). Despite the mutation profile bias introduced by SNP subtraction, removal of mutated reads at SNP sites reduced the cosine similarity between the SBS profiles of cases and controls to a mean of 0.982 using bootstrapping with 50 iterations (95% CI 0.982-0.983, FIG. 10D), compared to 0.999 when SNPs were retained (95% CI 0.999-0.999, FIG. 2F). These data suggest that SNP-subtracted data may be more suited to cancer classification, though SNP-retained data provides a more accurate signature profile.

### Example 5: Development of the Classification Method

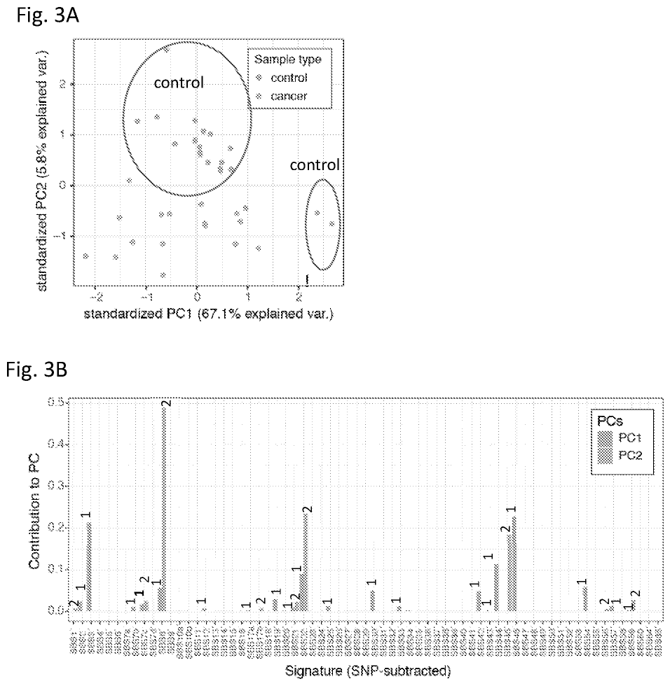

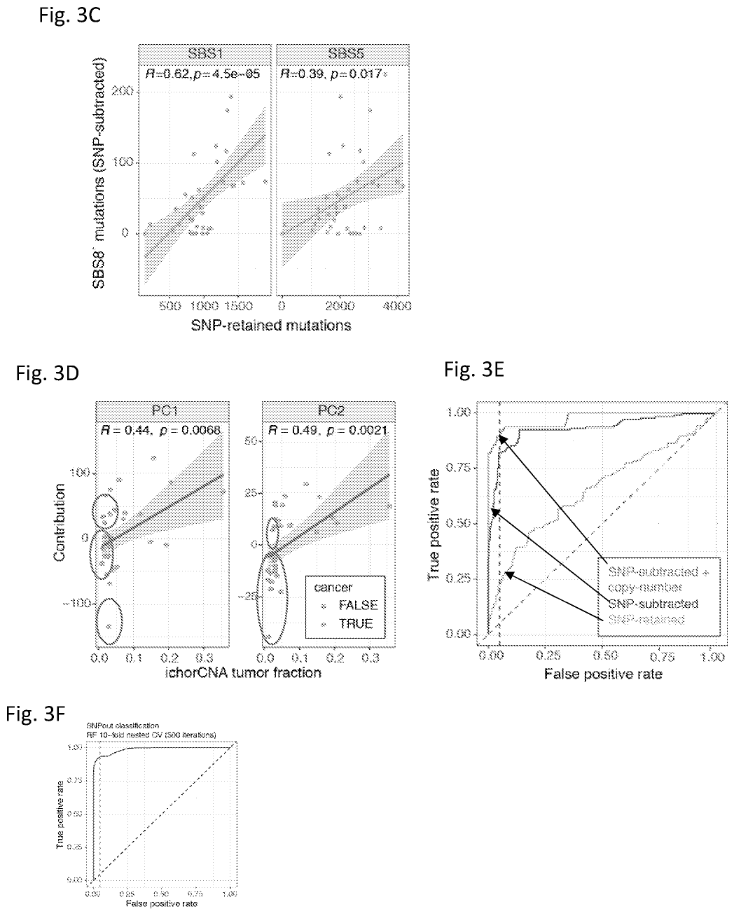

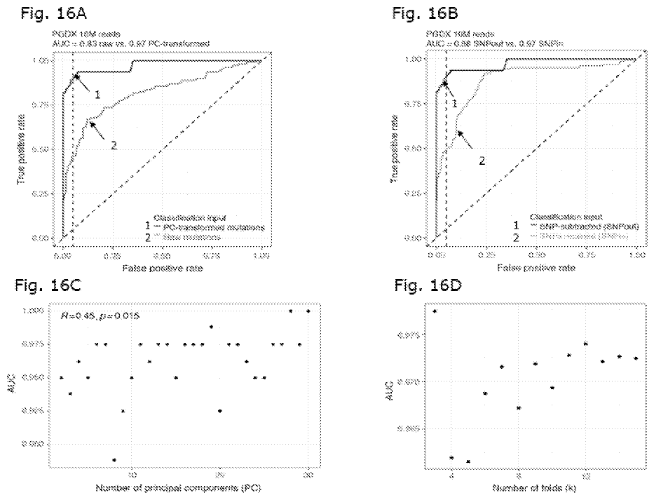

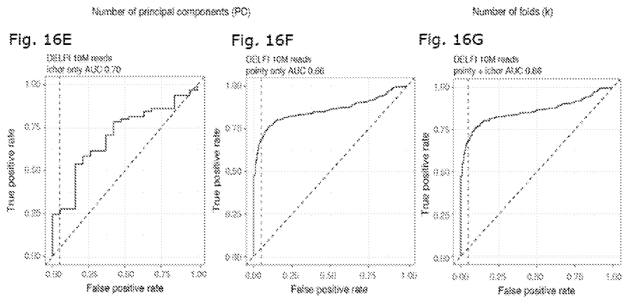

We first generated PCs for each of the samples, which correlated with ctDNA fraction (FIG. 3D). Classification of samples using PCs showed better performance than using the raw mutation count matrix (FIG. 16A), and showed better performance when SNPs were subtracted (FIG. 16B). We found that PCs contributing <1% of variance added limited information to the model, so these were dropped to minimize the risk of overfitting (FIG. 16C). 10-fold cross validation was used—with lower values of k, the AUC was more variable; with higher values of k, the AUC remained stable (FIG. 16D). Plasma WGS from the DELFI study were used and downsampled to 10M reads per sample. Sample classification was performed using ichor alone, AUC=0.70 (FIG. 16E); pointy alone, AUC=0.86 (FIG. 16F), and pointy and ichor combined, AUC=0.86 (FIG. 16G). Combining ichor and pointy provided no additional detection benefit over pointy alone in this dataset, though there was a non-significant increase in AUC in the PGDX cohort (PGDX pointy alone AUC=0.93 [95% CI 0.89-0.96], pointy and ichor AUC=0.97 [95% CI 0.94-1.00]). We applied the above settings, resulting in the classification performance shown in FIG. 3E.

### Example 6: Colorectal Cancer Detection

We next sought to classify samples into cancer vs. healthy based on SBS mutation profile. To maximize the signal-to-noise ratio of true cancer mutations, SNPs were removed. Then, SBS' (SNP-subtracted) mutation profiles underwent dimensionality reduction using Principal Component Analysis (PCA), and the principal components of SBS profiles (analogous to mutational signatures) were used for machine learning classification (Methods). PCA showed separation of cases and controls based on two Principal Components, particularly in PC2 (FIG. 3A). Using SNP-subtracted data, the signature contributions to PC1 and PC2 were assessed, which showed that SBS8′ was the greatest contributor to PC2 (FIG. 3B). As SNP-subtracted data were used, no mutations had been fitted to SBS1′ or SBS5′ (FIG. 10A). We assessed whether SBS8′ might represent aging mutations that were incorrectly fitted following SNP-subtraction by correlating SBS8′ with aging signatures (FIG. 3C), which showed SBS8′ was significantly correlated with SBS1 (p=4×10⁻⁵) and with SBS5 (p=0.017). PC1 and PC2 from SNP-subtracted data were both significantly correlated with ctDNA fraction (p<0.0068, FIG. 3D), consistent with the correlation previously shown between SNP-retained signatures and ichorCNA ctDNA fraction (FIG. 2D).

To classify samples as either cancer or healthy, we used an extreme gradient boosting (xgboost) machine learning model on each the PCA-transformed SBS profile of each sample, generating a Pointy score for each sample. We estimated performance characteristics using ten-fold cross validation repeated ten times. With SNPs subtracted, an AUC of 0.93 was reached (95% CI 0.89-0.96, FIG. 3E). Combining ichor ctDNA fraction in the model resulted in a non-significant improvement in AUC (0.97, 95% CI 0.94-1.00). To confirm the enhanced signal-to-noise ratio following removal of SNPs from Pointy data, classification was performed with SNPs retained, which showed an AUC of 0.65 (95% CI 0.59-0.71).

We next compared three other models for cancer detection using PCA-transformed input, including: random forest (RF), xgboost, support vector machine (SVM) and logistic regression. Nested 10-fold cross-validation was used (Methods). Across all models, with SNPs subtracted, a median AUC of 0.95 was reached (range 0.94-0.97, FIGS. 30B-30E), with RF performing best (AUC 0.97, 95% CI 0.93-1.00). Adding ichor ctDNA fraction to the model improved the AUC of the RF model to 0.99 (95% CI 0.98-1.00, FIG. 30C), which was selected for subsequent analyses. We confirmed this result with 10-fold cross-validation with 500 iterations (FIG. 3F). To assess the effect of downsampling, we iteratively downsampled the data to 10M reads 50 times, confirming a mean AUC of 0.97 (95% CI 0.96-0.98, FIG. 30F).

To confirm the enhanced signal-to-noise ratio following removal of SNPs from Pointy data, classification was performed using RF with SNPs retained, which showed an AUC of 0.74 (95% CI 0.64-0.87, FIG. 30G). We also tested the effect of error-suppression, i.e. requiring mutations to be supported in both F and R mate pairs vs. being supported in either F or R only, which showed AUC values of 0.98 vs. 0.93 (P=0.004, Wilcoxon test, FIG. 30H). Therefore, for subsequent analyses for cancer detection, we processed data by using (a) SNP-subtraction, (b) PC transformation, (c) mutations only in both F and R reads, and (d) detection using an RF model (Methods).

### Example 7: Signature Detection in Plasma Across Multiple Cancer Types

To validate this approach in an external dataset, we applied Pointy to the Cristiano et al.¹³ plasma WGS dataset to test this approach across multiple cancer types. This cohort consisted of stage I-IV NSCLC (n=37), stage I-III breast cancer (n=48), stage I-IV CRC (n=27), stage I-IV, 0 and X gastric cancer (n=27), stages I, III and IV ovarian cancer (n=26), stage I-III pancreatic cancer (n=34), and 227 individuals without cancer. By comparing sequencing read headers, samples were determined to be sequenced across multiple sequencing instruments (FIG. 11A). We focused on samples from one sequencer (HWI-D00837), which contained baseline samples from patients with stage I-IV non-small cell lung cancer (NSCLC, n=21), stage I-III pancreatic cancer (n=27) and stages I-IV and X gastric cancer (n=15, including one patient with stage X disease). The median number of sequencing reads per sample was 75.8×10⁶. Data were processed similarly to those in the discovery set, including GC normalization, and downsampled to 10M reads.

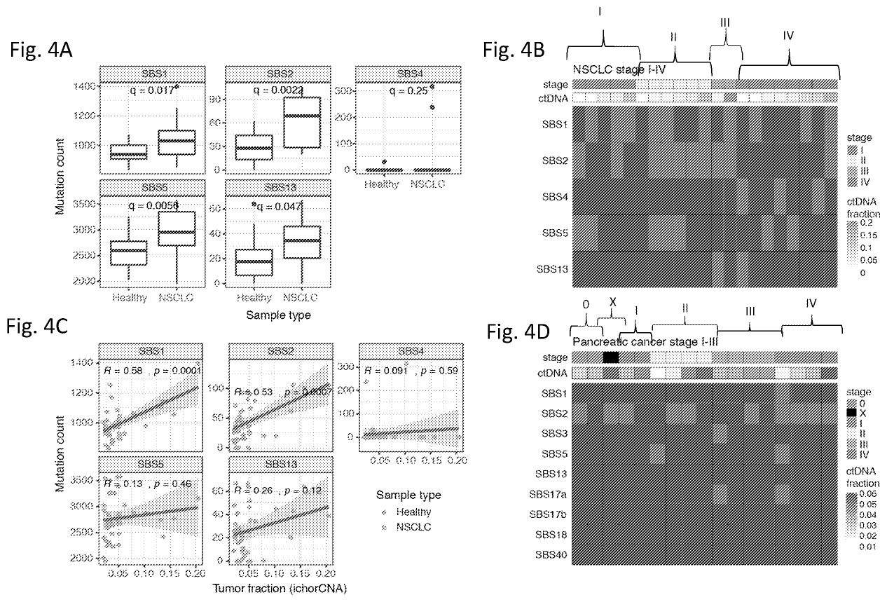

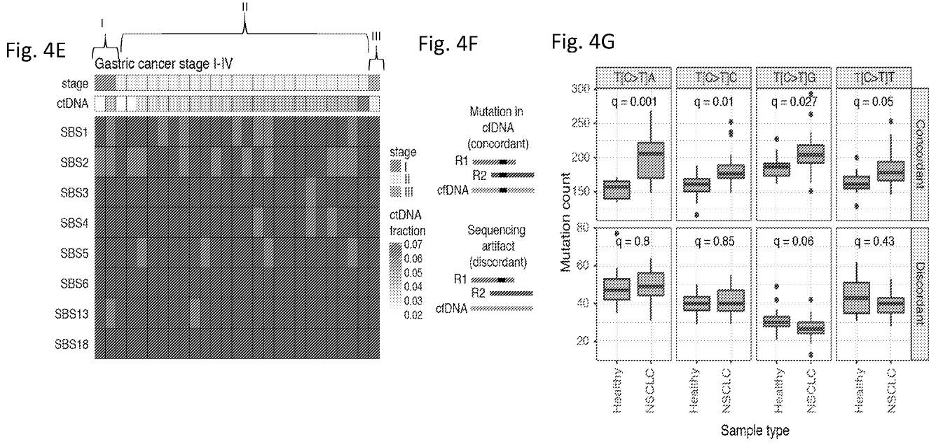

First, stage I-IV NSCLC samples were analyzed with SNPs retained. Signatures known to be associated with lung cancer² and tobacco exposure¹⁷ were assessed. Patients with NSCLC had significantly more mutations per sample than healthy individuals (median 10,321 vs. 9,590, p=5×10⁻⁴, two-tailed Wilcoxon test). Patient samples had significantly more aging and smoking signature mutations in plasma compared to healthy individuals (FIG. 4A). Aging and smoking signatures called at a specificity of 95% are shown per sample in FIG. 4B. 20 out of 21 samples (95.2%) had at least one signature called in plasma, with aging and APOBEC signatures being the most prevalent (in 14 out of 21 samples each). SBS4 was observed in 3 out of 21 (14.3%) of samples, and all three of these samples were from patients with stage IV disease. In patients with NSCLC, SBS1 and SBS2 were significantly correlated with ctDNA fraction, though other aging and smoking signatures were not correlated (FIG. 4C). To confirm the biological validity of these data, we compared NSCLC plasma signatures against those from CRC samples from the PGDX cohort. To compare across cohorts, background-subtraction normalization was performed for cancer samples relative to their respective controls (Supplementary Methods), which showed NSCLC samples had significantly greater SBS2 (APOBEC, q=0.00014) and SBS4 (smoking, q=0.00014) compared to CRC. In contrast, CRC samples showed significantly more SBS21 (MSI, q=0.001, FIG. 11B).

Patients with stage I-III pancreatic cancer and stages I-IV or X gastric cancer had low ctDNA detection rates using ichorCNA: 4 out of 27 (14.8%) and 3 out of 15 (17.6%) were detected with a specificity of 95%, respectively. In comparison, in patients with pancreatic cancer, SBS2 was detected using Pointy with 95% specificity in 11 out of 27 patients (40.7%), and aging signatures were detected in 5 out of 27 (18.5%, FIG. 4D). In patients with gastric cancer, 9 out of 15 (60.0%) patients had SBS2 detected with 95% specificity (FIG. 4E), whereas aging signatures were detected in 2 out of 15 (13.0%). For both of these cancer types, there was no significant correlation between SBS2 and ctDNA fraction (q>0.80, FIG. 11C), though the range of ctDNA fractions was small and below the detection threshold of the copy-number based used for ctDNA quantification (pancreatic cancer, range 0.022-0.065; gastric cancer, range 0.015-0.057).

The ratio between short (<150 bp) to long fragments (>150 bp) was assessed for both each cancer type. Both pancreatic and gastric cancer patients had significantly longer mutant fragments than healthy controls (p<2.5×10⁻⁵, FIG. 11D). In contrast, patients with NSCLC showed significantly shorter fragments than healthy controls (p=1.4×10⁻¹¹). This observed lengthening and shortening of mutant fragments is consistent with a previous report²⁵. Together, these data suggest the presence of APOBEC mutations in long cfDNA fragments, which could arise from either tumor cells or the microenvironment, though it is not possible to discern their specific origin using these data alone.

Given the prevalence of SBS2 mutations in the above Cristiano et al.¹³ sequencing data, we sought to measure per-signature noise for each sample. To quantify noise, we utilized the discordant mutations in the overlapping region of paired-end sequencing reads in each sample (FIG. 4F). Discordance in mutations between overlapping R1 and R2 reads likely arise from sequencing noise¹⁹, as true mutations would be present in both R1 and R2. The number of discordant mutations per sample was constant across each of the SBS contexts of SBS2 in patients with NSCLC compared to healthy individuals (q>0.06, FIG. 4G), suggesting that SBS2 calls are unlikely to arise from sequencing noise.

### Example 8: Aging Signatures in Healthy Individuals

Given the predominance of aging signatures in Pointy data, we explored the relationship of aging signatures with chronological age in healthy individuals. Individuals with cancer were not used for this analysis to eliminate tumor cells as a source of aging mutations. We expected the magnitude of any relationship to be small based on previous estimates of aging mutation rates¹⁷, combined with recent evidence for aging signatures varying between tissues²⁶.

Limited cohort analysis: three sequencing runs containing heathy individuals' plasma data from the Cristiano et al.¹³ study were used (n=139) to maximize the power of this analysis. Data were downsampled to 50M reads (1.5×) WGS, GC-normalized per batch and signatures fitted with SNPs retained. The age range of healthy individuals in this cohort was 50-75 years old, with a median age of 54. The read headers in these data lacked a unique sequencer identifier, and so we treated them as arising from different sequencers. Thus, signature contributions for each batch were normalized by taking the mean SBS contributions for the youngest individuals in each batch (aged 50, n=40), which were used to mean-center all data points in each batch (Supplementary Methods).

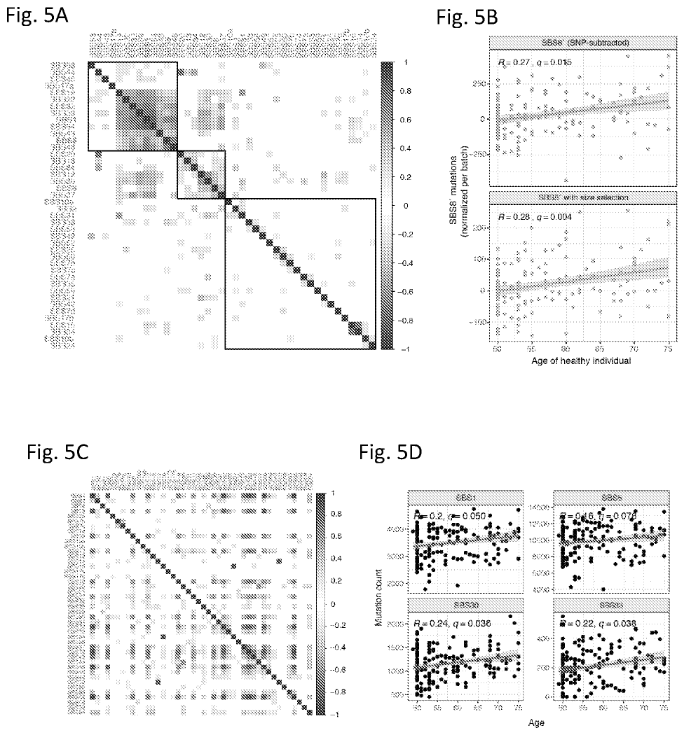

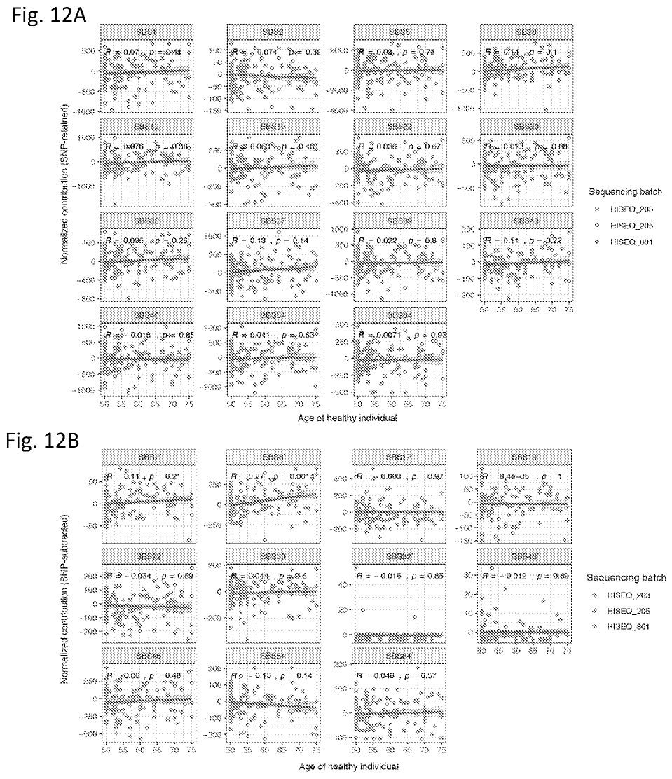

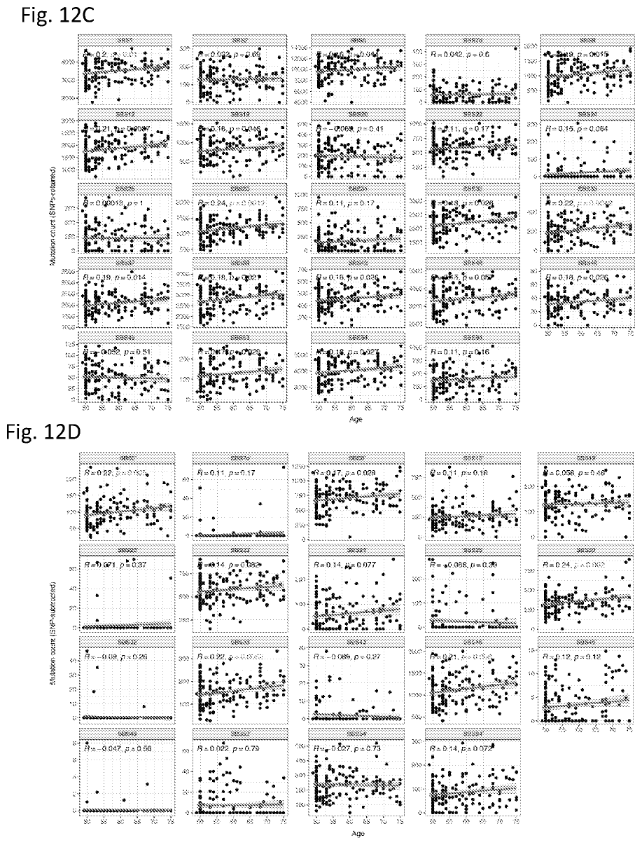

Signatures that were significantly correlated with SBS1 and SBS5 with SNPs-retained were identified as putative aging correlated signatures (FIG. 5A). These SBS1/SBS5-correlated signatures were compared against the chronological age of each healthy individual, however, none of these SNP-retained signatures showed significant correlation (FIG. 12A). When SNPs were subtracted, SBS8′ (SNP-subtracted) showed a small but significant correlation with age (Pearson r=0.24, q=0.015, FIG. 5B, upper panel). Each of the SNP-subtracted signatures tested are shown in FIG. 12B. Interestingly, in the previous CRC cohort, SBS8′ was identified to contribute to cancer detection (FIG. 3B) and was shown to correlate with aging signatures (FIG. 3C). Although preliminary, these data suggest that SBS8′ might represent aging mutations in healthy individuals that were erroneously fitted due to SNP-subtraction (FIG. 10A).

To assess the fragmentation pattern of mutant molecules in healthy individuals, size selection of short fragments (<150 bp) was performed. Size selection increased the magnitude and significance of the correlation (Pearson r=0.28, q=0.004, FIG. 5B, lower panel), indicating that aging mutations are in short cfDNA molecules, similar to the finding of ctDNA fragments being shorter than wild-type fragments in patients with cancer¹².

Larger cohort analysis. 159 heathy individuals' plasma data arising from the same sequencer from the Cristiano et al.¹³ study were used. Data were downsampled to 50M reads (1.5×) WGS, GC-normalized per batch and signatures fitted with SNPs retained. The age range of healthy individuals in this cohort was 49-75 years old, with a median age of 54.

Signatures that were significantly correlated with SBS1 using SNPs-retained were identified as putative aging-correlated signatures (FIG. 5C). These potential aging signatures were compared against the chronological age of each healthy individual. Following correction for multiple testing, SBS1, SBS30 and SBS33 showed significant correlation with chronological age (q≤0.05, selected signatures shown in FIG. 5D, all signatures shown in FIG. 12C).

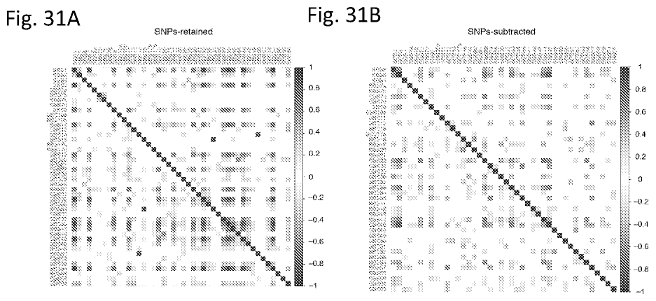

With SNP-subtracted data, multiple SBS1-correlated signatures showed significant correlation with chronological age (SBS2′, SBS30′, SBS33′ and SBS46′, Pearson r range=0.21-0.24, q<0.03), though no mutations fitted to SBS1′ in this case due to bias introduced by SNP-subtraction (FIG. 10). SNP-subtracted signatures are shown in FIG. 12D. SBS2′, SBS30′, SBS33′ and SBS46′ mutation counts were significantly correlated with one another (FIGS. 31A-31B). These data suggest that aging mutations may be detected in the plasma of healthy individuals, both in SBS1 and SBS1-correlated signatures, the latter is likely due to misattribution of mutations to other signatures due to SNP removal.

### Example 9: Cancer Classification in a Validation Cohort

For all cancer types in the individual batch from the Cristiano et al.¹³ cohort, cancer detection and classification of cancer type were performed using SNP-subtracted SBS profiles. ichorCNA ctDNA fractions were included in each model, as before. Samples were downsampled to 25M (0.75×) reads and nested 10-fold cross-validation was used, repeated 500 times (Methods).

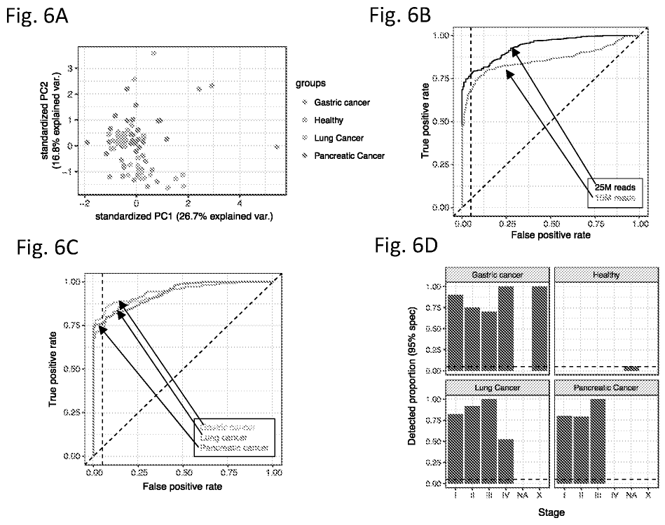

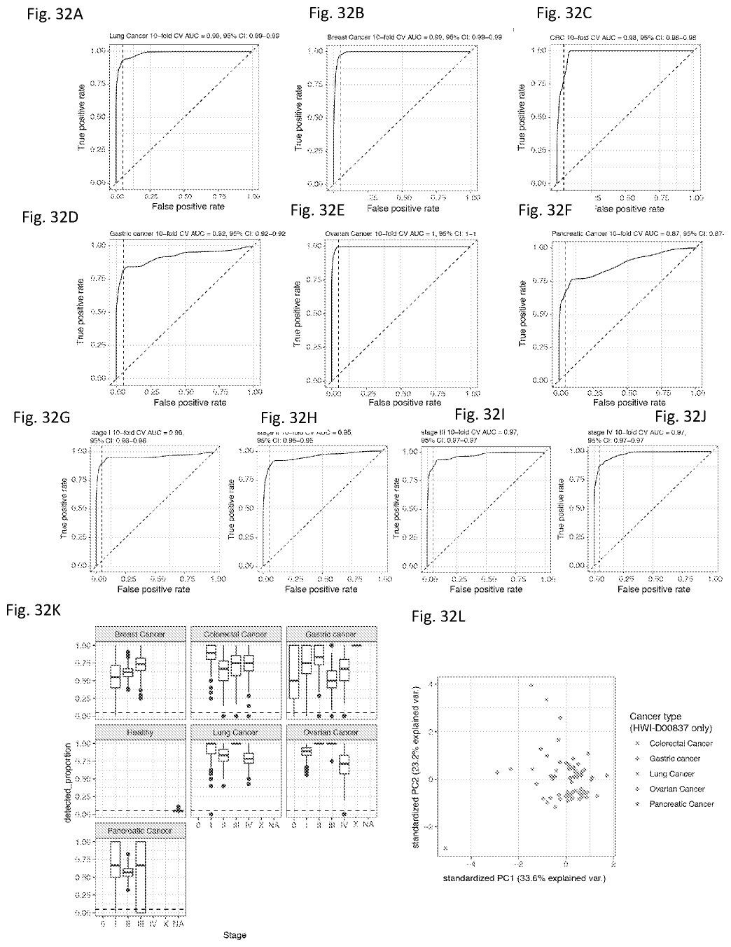

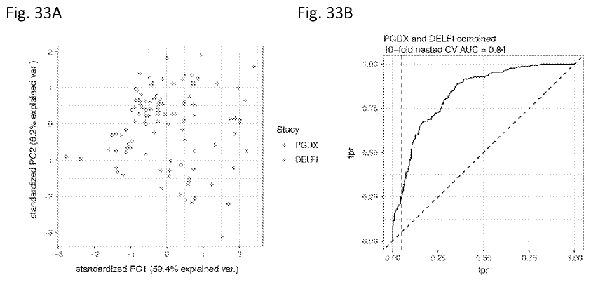

PCA showed differences between patients and healthy individuals, and also showed clustering of patients by cancer type (FIG. 6A). For cancer detection, the AUC with WGS with 10M reads (0.3×) was 0.89 with 10-fold cross validation, repeated 10 times (95% CI 0.86-0.91, FIG. 6B). To assess the impact of greater sequencing depth, cancer detection was repeated with WGS with 25M reads, which increased the AUC to 0.94 (95% CI 0.93-0.95, FIG. 6B). Across all stages, the AUC values were 0.99 for NSCLC (95% CI 0.99-0.99), 0.99 for breast cancer (95% CI 0.99-0.99), 0.98 for CRC (95% CI 0.98-0.98), 0.92 for gastric cancer (95% CI 0.92-0.92), 1.00 for ovarian cancer (95% CI 1.00-1.00), 0.87 for pancreatic cancer (95% CI 0.87-0.88). See FIGS. 32A-32F. Detection rates of patients across all stages was high (FIGS. 32G-J), as follows: stage I, AUC 0.96 (95% CI 0.96-0.98,); stage II, AUC 0.95 (95% CI 0.95-0.95); stage III, AUC 0.97 (95% CI 0.97-0.97); stage IV, AUC 0.97 (95% CI 0.97-0.97). Detection rates by stage and cancer type with specificity set to 95% are shown in FIG. 32K. Based on differences observed in PCI and PC2 between samples using PCA (FIG. 32L), we assessed whether samples could be classified into individual cancer types. We selected patient samples sequenced on the same sequencer from the DELFI study (n=70). Healthy samples were excluded. Classification to individual cancer types achieved an accuracy of 0.77 (95% CI 0.74-0.80), significantly above the no information rate (P<2×10⁻¹⁶).

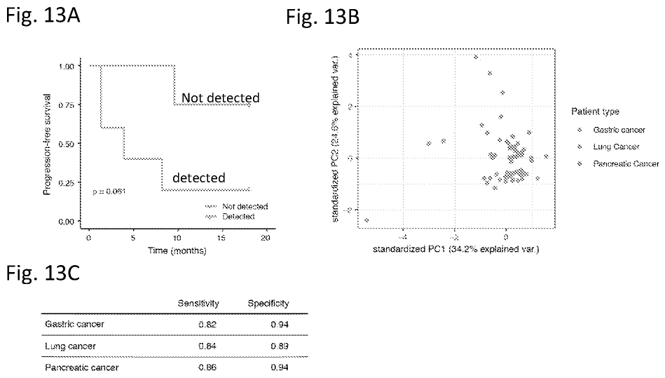

For patients with stage I-III disease, 41 out of 50 (82%) were detected with a specificity of 95%; the detection rates by stage are shown in FIG. 6D. In patients with stage IV disease, 8 out of 12 (67%) patients were detected. Interestingly, all of the non-detected samples were from patients with stage IV lung adenocarcinoma. To explore this result, ctDNA fractions for these samples using targeted sequencing were obtained¹⁸, which showed that of the samples not detected by Pointy, 3 out of 4 (75%) were undetected using targeted sequencing. Furthermore, using outcomes data by Cristiano et al.¹³, patients with NSCLC who had detected vs. undetected ctDNA using Pointy at baseline showed a median progression-free survival (PFS) of 3.9 months vs. median not reached with 18 months' follow-up (HR 6.8, p=0.06, FIG. 13A).

Given the separation in cancer types in PC1 and PC2 using PCA (FIGS. 6A and 13B), we assessed whether samples could be classified into individual cancer types (Methods). For this analysis, all cancer samples were included, regardless of ctDNA detection status. Healthy samples were not included. Classification to individual cancer types achieved a median sensitivity of 0.84 (range 0.82-0.86) with a median specificity of 0.94 (range 0.89-0.94, FIG. 13C).

Lastly, we assessed generalizability of this approach across cohorts, as patients with CRC were common to both cohorts. We identified evidence of batch effect affecting SNP-subtracted mutation profiles of healthy controls between the two studies (FIG. 33A), despite using quality filters and GC-bias correction. This may be due differences in sample collection location, as cases were collected across multiple academic sites, with controls in the former study sourced from a separate commercial site^{15,21}. To mitigate this, we pooled healthy and CRC patient samples across the two studies to allow training across batches. 10-fold nested CV was performed using RF, resulting in an AUC of 0.84 (FIG. 33B).

### Example 10: Classification to TMB Low Vs. High Using Pointy

Somatic mutations have the potential to generate non-self, immunogenic antigens. Tumors with a large number of somatic mutations, or tumor mutation burden (TMB), have been shown to respond to immune checkpoint blockade (ICB)³⁵. Mutational processes that result in high TMB can also contribute to ICB response. Microsatellite instability (MSI) and mismatch repair (MMR) deficiency also predict response ICB³⁶⁻³⁷. TMB is used across multiple cancer types for identification of patients who may benefit from ICB. A targeted plasma sequencing approach which analyzed microsatellite regions using hybrid-capture demonstrated specificity >99% and sensitivities of 78% and 67% for MSI and TMB-high, respectively²⁰. For patients in the same cohort who were treated with PD-1 blockade, MSI and TMB-high identified in pre-treatment plasma significantly predicted progression free survival (P<0.003).

Previous methods for quantifying TMB plasma rely confident mutation calls from matched tumor and normal sequencing data of sufficient depth^{20,32}. MSI identification may also be performed by comparing the lengths of microsatellites between cancer and normal³³, which may also be performed in plasma³⁴. Recently, by applying a personalized sequencing method, it was shown that despite limited depth, low-coverage WGS contains point mutation signal at patient-specific loci¹⁵. In this analysis, we developed an approach called Pointy to analyze genome-wide mutational signatures from plasma WGS at 0.3× for inexpensive TMB quantification and MSI classification for patient selection for ICB.

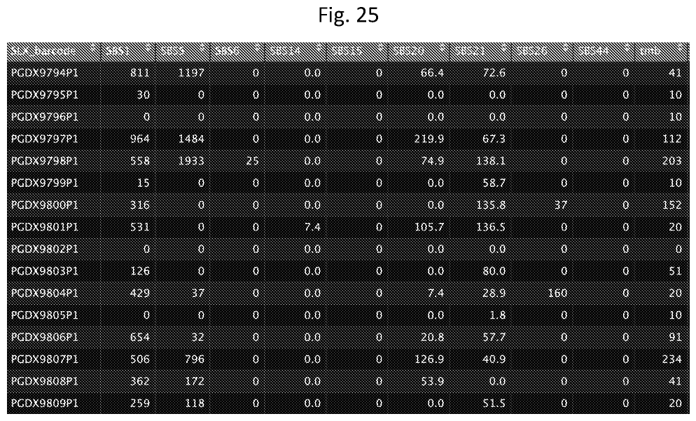

To test whether plasma signature contributions correlated with TMB, TMB was determined by targeted panel sequencing of plasma. A matrix of signature contributions and TMB values is shown in FIG. 25.

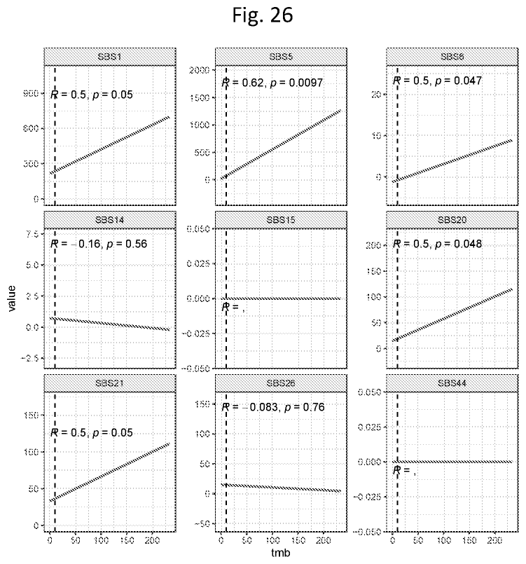

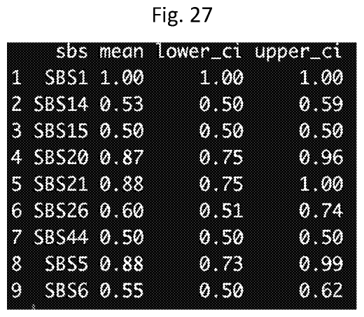

We found that SBS1 and SBS5 were significantly correlated with TMB (adjusted p<0.05, FIG. 25), but SBS20 and SBS21 were not (FIG. 26). Data were bootstrapped 25 times, and the AUC for classification of samples to high/low TMB based on a threshold of >10mut/mb was assessed for each SBS. The highest AUC for classification to high/low TMB was 1.00 (95% CI 1.00-1.00) for SBS1. The classification AUCs for all SBS signatures are shown in FIG. 27.

### Discussion

In this study, we identified mutational signatures in low-coverage plasma WGS from two independent data sets. Both exogenous and endogenous mutational processes were identified in plasma, including aging, smoking, APOBEC and MSI signatures. Circulating mutational signatures may be utilized for non-invasive signature profiling and cancer detection with high sensitivity and specificity. As such exposures (and their associated mutational signatures) may occur prior to cancer development, signature-based detection approaches might facilitate earlier cancer detection or help further define risk for developing cancer. In healthy individuals, an age-correlated mutational signature was identified in plasma, suggesting that interrogating the mutational processes that predate cancer might provide useful information.

In various embodiments, matched germline samples, which have the advantage of improving the scalability of the approach, may be used. Incorporating matched germline samples may improve sensitivity for low abundance circulating signatures. Additionally, in various embodiments, error-suppression may be used due to the low-coverage of the data. To mitigate sources of noise, data may be fitted to known SBS signatures rather than attempting signature discovery (thereby introducing additional variance), plus machine learning is leveraged for classification of samples within each batch. These data employed a limited number of mutational signatures, which were likely the most prevalent in somatic cells and thus the circulation. By comparing cases and controls within the same batch, differences in signature profile could be confidently attributed to cancer through signature detection with a specificity of 95%.

This analysis of low-coverage plasma WGS provides an insight into the possible array of pathological and physiological signatures that may be identified in cfDNA. These signatures, whose exposures may be operative both before and during cancer development¹, might be used for earlier cancer detection. Despite the low sequencing coverage utilized in this study, sensitive cancer detection was shown, enabling an inexpensive and scalable cancer detection approach. Moreover, improved profiling of physiological signatures in healthy individuals may enable the interrogation of cancer risk.

### REFERENCES

1. Stratton, M. R., Campbell, P. J. & Futreal, P. A. The cancer genome. Nature 458, 719-724 (2009).
2. Alexandrov, L. B. et al. The repertoire of mutational signatures in human cancer. Nature 578, 94-101 (2020).
3. Wan, J. C. M. et al. Liquid biopsies come of age: towards implementation of circulating tumour DNA. Nat Rev Cancer 17, 223-238 (2017).
4. Bronkhorst, A. J., Ungerer, V. & Holdenrieder, S. The emerging role of cell-free DNA as a molecular marker for cancer management. Biomol. Detect. Quantif 17, 100087 (2019).
5. Sun, K. et al. Plasma DNA tissue mapping by genome-wide methylation sequencing for noninvasive prenatal, cancer, and transplantation assessments. Proc. Natl. Acad. Sci. 112, E5503-E5512 (2015).
6. World Health Organization. Guide to Cancer—Guide to cancer early diagnosis. World Health Organization 48 (2017).
7. Newman, A. M. et al. Integrated digital error suppression for improved detection of circulating tumor DNA. Nat Biotechnol 34, 547-55 (2016).
8. Cohen, J. D. et al. Detection and localization of surgically resectable cancers with a multi-analyte blood test. Science. 359, 926-930 (2018).
9. Adalsteinsson, V. A. et al. Scalable whole-exome sequencing of cell-free DNA reveals high concordance with metastatic tumors. Nat. Commun. 8, 1324 (2017).
10. Chabon, J. J. et al. Integrating genomic features for non-invasive early lung cancer detection. Nature (2020). doi:10.1038/s41586-020-2140-0
11. Liu, M. C., Oxnard, G. R., Klein, E. A., Swanton, C. & Seiden, M. V. Sensitive and specific multi-cancer detection and localization using methylation signatures in cell-free DNA. Ann. Oncol. (2020). doi:10.1016/j.annonc.2020.02.011
12. Mouliere, F. et al. Enhanced detection of circulating tumor DNA by fragment size analysis. Sci. Transl. Med. 4921, 1-14 (2018).
13. Cristiano, S. et al. Genome-wide cell-free DNA fragmentation in patients with cancer. Nature 570, 385-389 (2019).
14. Lee-Six, H. et al. The landscape of somatic mutation in normal colorectal epithelial cells. Nature 574, 532-537 (2019).
15. Wan, J. C. M. et al. ctDNA monitoring using patient-specific sequencing and integration of variant reads. Sci. Transl. Med. 12, eaaz8084 (2020).
16. Chen, T. & Guestrin, C. XGBoost: A Scalable Tree Boosting System. in Proceedings of the 22nd ACM SIGKDD International Conference on Knowledge Discovery and Data Mining 785-794 (Association for Computing Machinery, 2016). doi:10.1145/2939672.2939785
17. Yoshida, K. et al. Tobacco smoking and somatic mutations in human bronchial epithelium. Nature 578, 266-272 (2020).
18. Phallen, J. et al. Direct detection of early-stage cancers using circulating tumor DNA. Sci. Transl. Med. 9, eaan2415 (2017).
19. Zviran, A. et al. Genome-wide cell-free DNA mutational integration enables ultra-sensitive cancer monitoring. Nat. Med. 26, 1114-1124 (2020).
20. Georgiadis, A. et al. Noninvasive detection of microsatellite instability and high tumor mutation burden in cancer patients treated with PD-1 blockade. Clin. Cancer Res. 25, 7024-7034 (2019).
21. Benjamini, Y. & Speed, T. P. Summarizing and correcting the GC content bias in high-throughput sequencing. Nucleic Acids Res. 40, 1-14 (2012).
22. Underhill, H. R. et al. Fragment Length of Circulating Tumor DNA. PLoS Genet. 12, 426-37 (2016).
23. Jamal-Hanjani, M. et al. Detection of Ubiquitous and Heterogeneous Mutations in Cell-Free DNA from Patients with Early-Stage Non-Small-Cell Lung Cancer. Ann. Oncol. 27, 862-7 (2016).
24. Jung, H., Bleazard, T., Lee, J. & Hong, D. Systematic investigation of cancer-associated somatic point mutations in SNP databases. Nat. Biotechnol. 31, 787-789 (2013).
25. Jiang, P. et al. Lengthening and shortening of plasma DNA in hepatocellular carcinoma patients. Proc. Natl. Acad. Sci. U.S.A. 112, E1317-E1325 (2015).
26. Afsari, B. et al. Supervised mutational signatures for obesity and other tissue-specific etiological factors in cancer. Elife 1-71 (2021).
27. Le, D. T. et al. Mismatch repair deficiency predicts response of solid tumors to PD-1 blockade. Science 357, 409-413 (2017).
28. Bolger, A. M., Lohse, M. & Usadel, B. Trimmomatic: a flexible trimmer for Illumina sequence data. Bioinformatics 30, 2114-2120 (2014).
29. Morgulis, A., Gertz, E. M., Schaffer, A. A. & Agarwala, R. WindowMasker: window-based masker for sequenced genomes. Bioinformatics 22, 134-141 (2006).
30. Blokzijl, F., Janssen, R., van Boxtel, R. & Cuppen, E. MutationalPatterns: Comprehensive genome-wide analysis of mutational processes. Genome Med. 10, 1-11(2018).
31. Chandrananda, D. et al. Investigating and correcting plasma DNA sequencing coverage bias to enhance aneuploidy discovery. PLoS One 9, (2014).
32. Koeppel, F., Blanchard, S., Jovelet, C., Genin, B., Marcaillou, C., Martin, E., Rouleau, E., Solary, E., Soria, J. C., André, F., et al. (2017). Whole exome sequencing for determination of tumor mutation load in liquid biopsy from advanced cancer patients. PLoS One 12, 1-14.
33. Niu, B., Ye, K., Zhang, Q., Lu, C., Xie, M., McLellan, M. D., Wendl, M. C., and Ding, L. (2014). MSIsensor: microsatellite instability detection using paired tumor-normal sequence data. Bioinformatics 30, 1015-1016.
34. Han, X., Zhang, S., Zhou, D. C., Wang, D., He, X., Yuan, D., Li, R., He, J., Duan, X., Wendl, M. C., et al. (2021). MSIsensor-ct: microsatellite instability detection using cfDNA sequencing data. Brief Bioinform.
35. Chan, T. A., Yarchoan, M., Jaffee, E., Swanton, C., Quezada, S. A., Stenzinger, A., and Peters, S. (2019). Development of tumor mutation burden as an immunotherapy biomarker: Utility for the oncology clinic. Ann. Oncol. 30, 44-56.
36. Le, D. T., Uram, J. N., Wang, H., Bartlett, B. R., Kemberling, H., Eyring, A. D., Skora, A. D., Luber, B. S., Azad, N. S., Laheru, D., et al. (2015). PD-1 Blockade in Tumors with Mismatch-Repair Deficiency. N. Engl. J. Med., 150530061707006.
37. Le, D. T., Durham, J. N., Smith, K. N., Wang, H., Bartlett, B. R., Aulakh, L. K., Lu, S., Kemberling, H., Wilt, C., Luber, B. S., et al. (2017). Mismatch repair deficiency predicts response of solid tumors to PD-1 blockade. Science 357, 409-413.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method comprising:
performing whole genome sequencing (WGS) on cell-free nucleic acids present in a sample comprising whole blood, plasma, and/or serum obtained from a subject to identify a plurality of single point mutations;
generating a subject sample dataset comprising a patient point mutation profile corresponding to the identified plurality of single point mutations;
applying a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and
storing, in one or more data structures, an association between the subject and the one or more classifications,
wherein the patient point mutation profile comprises a plurality of single base substitution contexts and, a label characterizing each single base substitution context,
wherein the subject sample dataset comprises single nucleotide polymorphisms (SNPs) and wherein the patient point mutation profile comprises at least one mutational signature, wherein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, SBS85, SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

2. (canceled)

3. (canceled)

4. (canceled)

5. The method of claim 1, wherein the at least one mutational signature has a mutation count of at least 10, at least 100, or at least 1000; or
wherein the one or more mutational signatures of the training dataset comprises a smoking signature, an UV light exposure signature, or a signature derived from mutagenic agents; or
wherein the one or more mutational signatures of the training dataset comprises an aging signature; or
wherein the one or more mutational signatures of the training dataset comprises an APOBEC (apolipoprotein B mRNA editing enzyme, catalytic polypeptide-like) signature.

6. (canceled)

7. (canceled)

8. The method of claim 1, further comprising removing single nucleotide polymorphisms (SNPs) from the subject sample dataset prior to applying the predictive model to the subject sample dataset and optionally performing principal component analysis (PCA) on the patient point mutation profile prior to applying the predictive model to the subject sample dataset.

9. (canceled)

10. (canceled)

11. (canceled)

12. (canceled)

13. The method of claim 1, wherein the one or more known conditions comprises a cancer; or
wherein the classification comprises a cancer type, or a cancer stage; or
wherein the classification comprises a risk for developing cancer; or
wherein the cohort of study subjects comprises cancer patients, and/or non-cancer patients; or
wherein the WGS has a depth between 0.3 and 1.5, or between 5.0 and 10.0; or
wherein the WGS has a depth of less than 2.0, less than 1.0 or less than 0.3; or
wherein WGS has a depth of greater than 1.0 or greater than 2.0, or greater than 30.0.

14. (canceled)

15. (canceled)

16. The method of claim 1, wherein the predictive model employs a gradient boosting machine learning technique, wherein the gradient boosting technique comprises an xgboost-based classifier; or.
wherein the predictive model employs a decision tree machine learning technique, optionally wherein the decision tree machine learning technique comprises a random forest classifier.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A computing device comprising a processor and a memory comprising instructions executable by the processor to cause the computing device to:
perform whole genome sequencing (WGS) on cell-free nucleic acids present in a sample comprising whole blood, plasma, and/or serum obtained from a subject to identify a plurality of single point mutations;
generate a subject sample dataset comprising a patient point mutation profile corresponding to the identified plurality of single point mutations;
apply a predictive model to the subject sample dataset to generate one or more classifications, the predictive model having been trained using a training dataset generated from sequence reads corresponding to cell-free nucleic acids from a cohort of study subjects with one or more known conditions, the training dataset comprising one or more mutational signatures characterizing the one or more known conditions of the study subjects in the cohort; and
store, in one or more data structures, an association between the subject and the one or more classifications
wherein the patient point mutation profile comprises a plurality of single base substitution contexts and a label characterizing each single base substitution context,
wherein the subject sample dataset comprises single nucleotide polymorphisms (SNPs) and
wherein the patient point mutation profile comprises at least one mutational signature, and wherein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, SBS85, SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. A method for identifying at least one somatic mutational signature in a subject comprising:
receiving, by a computing system comprising one or more processors, a whole genome sequencing (WGS) dataset generated by performing, using a next-generation sequencer (NGS), WGS on cell-free nucleic acids present in a sample comprising whole blood, plasma, and/or serum obtained from a subject;
generating, by the computing system, a conditioned dataset by performing a set of operations comprising alignment and GC normalization of sequence reads in the WGS dataset, wherein the WGS dataset is conditioned such that it retains at least a minimum percentage of single nucleotide polymorphisms (SNPs);
identifying in the conditioned WGS dataset, by the computing system, single point mutations in the sequence reads in the conditioned WGS dataset based on a comparison of the sequences reads in the conditioned WGS dataset with a reference genome;
generating, by the computing system, based on the identified single point mutations, a single base substitutions (SBS) dataset comprising an SBS matrix with a frequency for each mutational variant in a set of SBS variants, wherein the set of SBS variants comprises 96 different contexts, each context corresponding to a unique 3 base pair (bp) combination of a mutated base and two adjacent bases on opposing sides of the mutated base; and
applying, by the computing system, a signature fitting technique to the SBS matrix to generate a point mutation profile that is indicative of at least one mutational signature detected in the cell-free nucleic acids present in the sample.

89. The method of claim 88, further comprising generating, by the computing system, a correlation score for the point mutation profile for one or more clinical metrics, optionally wherein the one or more clinical metrics comprises microsatellite instability (MSI), tumor mutation burden (TMB) and/or mutation count per signature.

90. (canceled)

91. (canceled)

92. (canceled)

93. The method of claim 89, further comprising administering to the subject a treatment based on the generated correlation score, optionally wherein the treatment comprises immune checkpoint blockade (ICB) therapy, optionally wherein the ICB therapy comprises one or more of a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

94. (canceled)

95. The method of claim 88, wherein the sample is a first sample taken prior to a treatment, and wherein the method further comprises:
receiving, by the computing system, a second WGS dataset generated by performing WGS on cell-free nucleic acids present in a second sample comprising whole blood, plasma, and/or serum obtained from the subject following the treatment;
generating, by the computing system, a second conditioned dataset by performing a set of operations comprising alignment and GC normalization of sequence reads in the second WGS dataset, wherein the second WGS dataset is conditioned such that it retains at least the minimum percentage of SNPs;
identifying in the second conditioned dataset, by the computing system, single point mutations in the sequence reads in the second conditioned dataset based on a second comparison of the sequences reads in the second conditioned dataset with the reference genome;
generating, by the computing system, based on the identified single point mutations, a second SBS dataset comprising a second SBS matrix with a frequency for each mutational variant in the set of SBS variants; and
applying, by the computing system, the signature fitting technique to the second SBS matrix to generate a second point mutation profile that is indicative of at least one mutational signature detected in the cell-free nucleic acids present in the second sample.

96. The method of claim 95, further comprising
generating, by the computing system, a second correlation score for the second point mutation profile with respect to at least one of the one or more clinical metrics; or
administering the treatment after the first sample is obtained from the subject.

97. (canceled)

98. The method of claim 95, further comprising comparing, by the computing system, the first point mutation profile with the second point mutation profile to determine an effect of the treatment on a disease phenotype, optionally wherein
the second point mutation profile lacks a mutational signature identified in the first point mutation profile, and wherein the effect indicates a decrease in a severity or duration of the disease phenotype in the subject; or
wherein the treatment is a first treatment, and wherein the method further comprises determining, by the computing system, a second treatment based on the effect of the first treatment.

99. (canceled)

100. (canceled)

101. The method of claim 98, further comprising administering the second treatment for the disease phenotype.

102. The method of claim 88, wherein the minimum percentage of SNPs retained is 25 percent, 50 percent, 75 percent or 95 percent.

103. The method of claim 98, wherein the disease phenotype is a cancer.

104. The method of claim 88, wherein the at least one mutational signature comprises one or more of SBS1, SBS2, SBS3, SBS4, SBS5, SBS6, SBS7a, SBS7b, SBS7c, SBS7d, SBS8, SBS9, SBS10a, SBS10b, SBS10d, SBS11, SBS12, SBS13, SBS14, SBS15, SBS16, SBS17, SBS17a, SBS17b, SBS18, SBS19, SBS20, SBS21, SBS22, SBS23, SBS24, SBS25, SBS26, SBS27, SBS28, SBS29, SBS30, SBS31, SBS32, SBS33, SBS34, SBS35, SBS36, SBS37, SBS38, SBS39, SBS40, SBS41, SBS42, SBS43, SBS44, SBS45, SBS46, SBS47, SBS48, SBS49, SBS50, SBS51, SBS52, SBS53, SBS54, SBS55, SBS56, SBS57, SBS58, SBS59, SBS60, SBS84, SBS85, SBS87, SBS88, SBS90, SBS92, SBS93, SBS94, SBS95, SBS96, SBS97, SBS98, SBS99, SBS100, SBS101, SBS102, SBS103, SBS104, SBS105, SBS106, SBS107, SBS108, SBS109, SBS110, SBS111, SBS112, SBS113, SBS114, SBS115, SBS116, SBS117, SBS118, SBS119, SBS120, SBS121, SBS122, SBS123, SBS124, SBS125, SBS126, SBS127, SBS128, SBS129, SBS130, SBS131, SBS132, SBS133, SBS134, SBS135, SBS136, SBS137, SBS138, SBS139, SBS140, SBS141, SBS142, SBS143, SBS144, SBS145, SBS146, SBS147, SBS148, SBS149, SBS150, SBS151, SBS152, SBS153, SBS154, SBS155, SBS156, SBS157, SBS158, SBS159, SBS160, SBS161, SBS162, SBS163, SBS164, SBS165, SBS166, SBS167, SBS168, and SBS169.

105. The method of claim 88, wherein the at least one mutational signature has a mutation count of at least 10, at least 100 or at least 1000.

106. The method of claim 88 wherein the at least one mutational signature comprises a smoking signature, an ultraviolet (UV) light exposure signature, a signature derived from mutagenic agents, an aging signature, and/or an APOBEC (apolipoprotein B mRNA editing enzyme, catalytic polypeptide-like) signature.

107. The method of claim 88, wherein the WGS has a depth between 0.3 and 1.5; or
wherein the WGS has a depth between 5.0 and 10.0; or
wherein the WGS has a depth of less than 2.0, less than 1.0, or less than 0.3
wherein WGS has a depth of greater than 1.0, greater than 2.0, or greater than 30.0.

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)
